(19) United States
(12) Patent Application Publication
(10) Pub. No.: US 2016/0271143 A1
(43) Pub. Date: Sep. 22, 2016

(54) ESTROGEN RECEPTOR MODULATORS FOR REDUCING BODY WEIGHT

(71) Applicant: RosScreening, Inc., Princeton, NJ (US)

(72) Inventors: Alexey G. RYAZANOV, Princeton, NJ (US); Alexander V. CHIKUNOV, Princeton, NJ (US)

(21) Appl. No.: 14/883,979

(22) Filed: Oct. 15, 2015

Related U.S. Application Data
(63) Continuation of application No. 14/369,484, filed on Jun. 27, 2014, now abandoned.
(60) Provisional application No. 61/581,921, filed on Dec. 30, 2011.

(51) Int. Cl. A61K 31/565; A61K 45/06; A61K 31/566; A61K 31/138; A61K 31/567; A61K 31/05; A61K 31/4535

(52) U.S. Cl. CPC A61K 31/565; A61K 31/05; A61K 45/06; A61K 31/4535; A61K 31/138; A61K 31/567; A61K 31/566

(57) ABSTRACT

The present invention relates to a method of reducing the body weight of a subject by administering an effective amount of an estrogen receptor modulator (ERM), optionally, in combination with an anti-obesity or weight loss agent.

This application claims the benefit of U.S. Provisional Patent Application No. 61/581,921, filed Dec. 30, 2011, which is hereby incorporated by reference.

### FIELD OF THE INVENTION

The present invention relates to a method of reducing the body weight of a subject by administering an effective amount of an estrogen receptor modulator (ERM), optionally, in combination with an anti-obesity or weight loss agent.

### BACKGROUND

Obesity has become a major health problem in the United States and other developed nations. In the United States, more than half of the adult population is considered overweight or obese. The incidence of obesity in children is also growing rapidly in many countries. Obesity is a major risk factor for cardiovascular disease, stroke, insulin resistance, type 2 diabetes, liver disease, neurodegenerative disease, respiratory diseases and other severe illnesses, and has been implicated as a risk factor for certain types of cancer including breast and colon cancer. Aside from its impacts on physical health, obesity has significant adverse effects on quality of life and psychological well-being. The incidence of obesity, already high, is likely to grow as a result of increasingly sedentary lifestyles in many countries. In addition, certain widely used psychiatric drugs, notably atypical antipsychotics, are associated with weight gain and increased risk of diabetes. Since these drugs must be used chronically to achieve adequate control of psychiatric symptoms, and treatment compliance in patients with mental disorders is frequently poor, these side effects present both a barrier to compliance and a significant additional health risk to patients.

Although it is well established that weight loss can be achieved through reduced caloric intake and increased physical activity, obesity has continued to be an intractable problem in Western countries, especially in the United States. The discovery of safe and effective drugs to induce weight loss has been a major research goal for decades. However, to date the drugs that have shown efficacy have been burdened with significant side effects or have shown only modest efficacy. For example, amphetamines have been used effectively as appetite suppressants but have a strong risk of dependence along with other side effects.

Accordingly, there is a significant need for new anti-obesity treatments. There is also a need for anti-obesity treatments with limited side effects that may be safely used in combination with other drugs that are in common use in obese patients, such as antidiabetic drugs, antihypertensive drugs, cholesterol-reducing agents, and insulin. Thus, agents that can be used for the treatment of obesity would represent a significant advance.

### SUMMARY OF THE INVENTION

The present invention relates to a method of reducing the body weight of a subject comprising administering an effective amount of an estrogen receptor modulator (ERM), such as an estrogen receptor agonist. Advantageously, it has been found that ERMs can reduce body weight without a substantial concomitant reduction in lean mass, substantial reduction in food consumption, or reduction in bone mineral density. It has also been found that ERMs restore serum glucose levels in subjects having elevated glucose levels and improve glucose tolerance.

Another embodiment is a method of reducing fat stored in adipose tissue without a substantial reduction in lean mass. The method includes administering an effective amount of an ERM.

Yet another embodiment is a method of treating obesity in a subject by administering an effective amount of an ERM.

Yet another embodiment is a method of reducing the risk of obesity or weight gain (or inhibiting or suppressing weight gain) in a subject by administering an effective amount of an ERM. In a preferred embodiment, the subject has a propensity or predisposition to become overweight or obese. In another embodiment, the subject is not overweight or obese.

Yet another embodiment is a method of reducing body weight, and/or inhibiting or suppressing weight gain, in a subject being administered a drug associated with weight gain (such as an atypical antipsychotic). The method comprises administering a therapeutically effective amount of an ERM (e.g., a SERM such as raloxifene or tamoxifen) to reduce body weight, and/or inhibit or suppress weight gain.

Yet another embodiment is a method of reducing a subject's weight by administering to the subject an ERM in an amount sufficient to reduce the subject's weight, where the subject has been identified (or diagnosed) as (i) being overweight or obese; and (ii) not having diabetes.

Yet another embodiment is a method of reducing the risk of a metabolic syndrome (such as type II diabetes) in a subject by administering to the subject an effective amount of an ERM. In one embodiment, the method includes administering an amount of the ERM effective to reduce the body weight of the subject.

Yet another embodiment is a method of treating a metabolic syndrome (such as type I diabetes or type II diabetes) in a subject by administering to the subject an effective amount of an ERM. In one embodiment, the method includes administering an amount of the ERM effective to reduce the body weight of the subject.

Yet another embodiment is a method of treating hyperglycemia in a subject (e.g., a subject suffering from diabetes, such as type I or type TT diabetes) by administering to the subject a therapeutically effective amount of an ERM. It has been found that ERMs restore basal serum glucose levels in hyperglycemic subjects. In addition, the subject may also be administered one or more other antidiabetic agents, such as insulin and insulin sensitizers.

Yet another embodiment is a method of treating impaired glucose tolerance or improving glucose tolerance in a subject having elevated glucose levels (e.g., a subject suffering from diabetes) by administering to the subject a therapeutically effective amount of an ERM. For example, a sufficient amount of ERM may be administered to control a subject's blood glucose level between 7.8 and 11.1 mmol/L (140-200 mg/dL).

The present invention also relates to the use of one or more ERMs in combination with other anti-obesity or weight loss agents. The ERM can enhance the weight loss and/or anti-obesity activity of the anti-obesity or weight loss agent. Accordingly, in the aforementioned methods, an effective amount of a combination of an ERM and an anti-obesity or weight loss agent can be used in lieu of an effective amount of the ERM alone. For example, one embodiment is a method of reducing the body weight of a subject comprising administering an effective amount of a combination of an ERM and an anti-obesity or weight loss agent. The ERM and anti-obesity or weight loss agent may be administered together in a single oral dosage form, co-administered at the same time (simultaneously) or near the same time in separate oral dosage forms, or at different times during the same day. A lower amount of ERM can be used in the co-therapy then the aforementioned monotherapy due to the action of the anti-obesity or weight loss agent.

Yet another embodiment is a kit comprising (i) one or more oral dosage forms of an ERM (e.g., raloxifene hydrochloride), and (ii) one or more dosage forms of the anti-obesity or weight loss agent (e.g., orlistat).

Discussed below are various embodiments of the aforementioned methods.

The ERM can be a selective estrogen receptor modulator (SERM). The ERM can be, for example, an estrogen receptor α modulator, an estrogen receptor β modulator, or a GPR-30 modulator. In one preferred embodiment, the ERM is an estrogen receptor agonist. For instance, the ERM can be an estrogen receptor α agonist, an estrogen receptor β agonist, or a GPR-30 agonist. The ERM can be a natural or synthetic estrogen, or a phytoestrogen (e.g., daidzein, formononetin, genistein, biochanin A, coumestrol, 4′-methoxycoumestrol, repensol, and trifoliol).

In one embodiment, the ERM is an organic compound which either possesses, or can be metabolized into a compound containing, at least one phenolic moiety (e.g., two or more phenolic moieties). In one particular embodiment, the organic compound (or its active metabolite) contains two phenolic moieties which are separated by a saturated or unsaturated moiety (e.g., a moiety which has one or two atoms between the two phenolic moieties) sufficient to achieve binding of the compound, or metabolized compound, to the estrogen receptor. Suitable chemical families which contain at least one phenolic moiety or nascent phenolic moiety include, but are not limited to, steroidals, diphenylethylenes, triphenylethylenes, diphenylethanes, triphenylethanes, benzothoiophenes, benzopyrans, flavones, isoflavones, di-phenyl pyrazoles, tri-phenyl pyrazoles, coumestins, benzothiaphenes, benzofurans, dibenzazulenes, benzazulenes, benzopyrroles, and tetrahydronaphthylenes. The phenyl ring of a phenolic moiety may be fused to another ring system (e.g., as in a benzothiophene ring system). In addition to the phenolic moiety, or nascent phenolic moiety, the compound is sufficiently lipophilic to achieve binding to the estrogen receptor. Nascent phenolic moieties are those moieties which upon metabolism or other chemical modification process in the body (such as oxidation, hydrolysis or nucleophilic substitution) yield a phenolic moiety. Nascent phenolic moieties include, but are not limited to, hydrogen, alkoxy, acyloxy, sufonyloxy, labile halogen or any other group which can be converted in the body to a phenolic moiety via chemical or biological means. These organic compounds may be in the form of a pharmaceutically acceptable salt.

Suitable ERMs include, but are not limited to, raloxifene, hexestrol, diethylstilbestrol, tamoxifen, clomiphene, femarelle, ormeloxifene, toremifene, lasofoxifene, 17β-estradiol, 17α-ethynyl estradiol, estrone, ethisterone, dienestrol, an analog thereof, a pharmaceutically acceptable salt of any of the foregoing, or any combination of any of the foregoing. Suitable analogs include those which retain phenolic or nascent phenolic moieties of the parent compound, and have sufficient lipophilicity to achieve binding to an estrogen receptor.

In one embodiment, the ERM is raloxifene or a pharmaceutically acceptable salt thereof. In another embodiment, the ERM is hexestrol or a pharmaceutically acceptable salt thereof. In yet another embodiment, the ERM is diethylstilbestrol or a pharmaceutically acceptable salt thereof. In yet another embodiment, the ERM is clomiphene or a pharmaceutically acceptable salt thereof. In yet another embodiment, the ERM is lasofoxifene or a pharmaceutically acceptable salt thereof. In yet another embodiment, the ERM is toremifene or a pharmaceutically acceptable salt thereof. In yet another embodiment, the ERM is estrone or a pharmaceutically acceptable salt thereof. In yet another embodiment, the ERM is 17β-estradiol or a pharmaceutically acceptable salt thereof. In yet another embodiment, the ERM is dienestrol or a pharmaceutically acceptable salt thereof.

In yet another embodiment, the ERM is tamoxifen or a pharmaceutically acceptable salt thereof. The tamoxifen may be administered with an agent which inhibits or prevents the in vivo conversion of tamoxifen to 4-hydroxytamoxifen and/or 3-hydroxytamoxifen. Tamoxifen is converted in the body to 4-hydroxytamoxifen by CYP 2D6, 3A4, 2C19, 2C9, and/or 2B6 (Sridar et al., Drug Metab Dispos 40:2280-2288). In the present invention, the tamoxifen may be administered with an inhibitor of CYP 2D6, 3A4, 2C19, 2C9, and/or 2B6. In one preferred embodiment, the tamoxifen is administered with an inhibitor of CYP 2D6.

The ERM can be administered orally, transdermally, intraarterially or intravenously. In one preferred embodiment, the ERM is administered orally. In another embodiment, the ERM is administered intravenously. In yet another embodiment, the ERM is administered transdermally (e.g., via a patch).

In one embodiment, the amount of ERM administered is greater than that typically administered to treat other known disorders for which it is used, such as osteoporosis (i.e., at a supra-therapeutic dose).

In some embodiments, the pharmaceutically effective amount is administered in a single dose per day. In some embodiments, the pharmaceutically effective amount is administered in two or more doses per day.

In one embodiment, the food consumption of the subject is not reduced during the treatment.

In one embodiment, the subject has excess body fat. For example, the subject may be overweight or obese, or exhibit one or more symptoms of obesity. The subject's body mass index (BMI) prior to treatment may be from 25 kg/m² to 30 kg/m². For obese subjects, the obesity may be class I obesity. The subject's BMI may be from 30 kg/m² to 35 kg/m². The subject's obesity may also be class II obesity. The subject's BMI may be from 35 kg/m² to 40 kg/m². The subject's obesity may be class III obesity. The subject's BMI may be from 40 kg/m² to 80 kg/m².

In another preferred embodiment, the subject is not overweight or obese but has a propensity (or disposition) to become overweight or obese.

In some variations of one or more of the above embodiments, the method reduces a subject's weight by 1%, 2%, 3%, 4%, 5%, 6%, 7%, 8%, 9%, 10%, 11%, 12%, 13%, 14%, 15%, 16%, 17%, 18%, 19%, 20%, 25%, 30%, 35%, 40%, 45% 50%, 55%, 60% or 65%.

In some variations of one or more of the above embodiments, the weight of the subject has been measured or will be measured. For example, in some embodiments, the weight of the subject has been measured prior to administering the ERM and will be measured after administering the ERM.

In some variations of one or more of the above embodiments, the BMI of the subject has been measured or will be measured. For example, in some embodiments, the BMI of the subject has been measured prior to administering the ERM and will be measured after administering the ERM.

### BRIEF DESCRIPTION OF THE DRAWINGS

FIGS. 1-5 are graphs showing the change in body weight, water intake, food intake, fat, and lean mass, respectively, for mice treated with diethylstilbestrol as described in Example 1 (Phase 1).

FIGS. 6-9 are graphs showing the change in body weight, water intake, fat, and lean mass, respectively, for mice treated with hexestrol as described in Example 1 (Phase 1).

FIGS. 10-14 are graphs showing the change in body weight, water intake, food intake, fat, and lean mass, respectively, for mice treated with raloxifene as described in Example 1 (Phase 1).

FIGS. 15-19 are graphs showing the change in body weight, water intake, food intake, fat, and lean mass, respectively, for mice treated with tamoxifen as described in Example 1 (Phase 1).

FIGS. 20-24 are graphs showing the change in body weight, water intake, food intake, fat, and lean mass, respectively, for mice treated with clomiphene as described in Example 1 (Phase 1).

FIGS. 25-29 are graphs showing the change in body weight, water intake, food intake, fat, and lean mass, respectively, for mice treated with diethylstilbestrol as described in Example 1 (Phase 2).

FIGS. 30-33 are graphs showing the change in body weight, water intake, fat, and lean mass, respectively, for mice treated with hexestrol as described in Example 1 (Phase 2).

FIGS. 34-38 are graphs showing the change in body weight, water intake, food intake, fat, and lean mass, respectively, for mice treated with raloxifene as described in Example 1 (Phase 2).

FIGS. 39-43 are graphs showing the change in body weight, water intake, food intake, fat, and lean mass, respectively, for mice treated with tamoxifen as described in Example 1 (Phase 2).

FIGS. 44-53 are bar graphs showing the number of mice which gained or lost varying weight amounts when treated with diethylstilbestrol, hexestrol, raloxifene, tamoxifen, clomiphene (in particular clomiphene citrate), 17β-estradiol, 17α-ethynyl estradiol, estrone, or ethisterone as described in Example 2.

FIGS. 54 and 55 are graphs showing the change in body weight over time in lean mice placed on a high fat diet and treated with diethylstilbestrol, hexestrol, raloxifene, tamoxifen, and clomiphene as described in Phase 1 of Example 1.

FIG. 56 is a graph showing the change in food intake over time in lean mice placed on a high fat diet and treated with diethylstilbestrol, hexestrol, raloxifene, tamoxifen, and clomiphene as described in Phase 1 of Example 1.

FIG. 57 is a bar graph showing the change in fat and lean mass for lean mice placed on a high fat diet and treated with diethylstilbestrol, hexestrol, raloxifene, tamoxifen, and clomiphene after 8 weeks as described in Phase 1 of Example 1.

FIGS. 58 and 59 are graphs showing the change in body weight over time in obese mice treated with diethylstilbestrol, hexestrol, raloxifene, tamoxifen, and clomiphene as described in Phase 2 of Example 1.

FIG. 60 is a graph showing the change in food intake over time in obese mice treated with diethylstilbestrol, hexestrol, raloxifene, tamoxifen, and clomiphene as described in Phase 2 of Example 1.

FIG. 61 is a bar graph showing the change in fat and lean mass for obese mice treated with diethylstilbestrol, hexestrol, raloxifene, tamoxifen, and clomiphene and control mice after 8 weeks as described in Phase 2 of Example 1.

FIG. 62 is a graph showing the change in body weight in mice for a control group and groups treated with estrone, diethylstilbestrol, hexestrol, raloxifene, tamoxifen, clomiphene, 17β-estradiol, and 17α-ethynyl estradiol as described in Example 2.

FIGS. 63-66 are graphs showing the change in body weight in mice (by absolute weight change or percentage of body weight) for a control group and groups treated with hexestrol, raloxifene, tamoxifen, or clomiphene in water (FIGS. 63 and 64) or feed (FIGS. 65 and 66) as described in Example 3.

FIG. 67 is a graph showing the change in food intake over time in obese mice treated with hexestrol, raloxifene, tamoxifen, and clomiphene in water and control mice as described in Example 3.

FIGS. 68 and 69 are bar graphs showing the change in fat and lean mass for obese mice treated with hexestrol, raloxifene, tamoxifen, and clomiphene in water (FIG. 68) or feed (FIG. 69) and control mice after 8 weeks as described in Example 3.

FIGS. 70 and 71 are graphs of the serum glucose concentrations over time of control mice or mice treated with 1 mg/kg/day of hexestrol, following injection with an intraperitoneal glucose solution (FIG. 70) or administration of an oral glucose solution (FIG. 71) as described in Example 3.

FIG. 72 is a graph showing the change in body weight (as a percentage of body weight) over time in obese mice treated with lasofoxifene, toremifene, estrone, 17β-estradiol, dienestrol, and hexestrol as described in Example 4.

FIG. 73 is a graph showing the change in body weight (as a percentage of body weight) over time in obese mice treated with 0.125, 0.25, 0.5, 1, and 2 mg/kg/day of hexestrol as described in Example 4.

### DETAILED DESCRIPTION OF THE INVENTION

### Definitions

The term “estrogen receptor modulator” (ERM) includes compounds that can bind and change the activity of an estrogen receptor. These include, but are not limited to, natural and synthetic estrogens. A preferred class of ERMs are estrogen receptor agonists (e.g., estrogen receptor α agonists, an estrogen receptor β agonists, and GPR-30 agonists).

The phrase “glucose tolerance” refers to the ability of a subject to control the level of plasma glucose and/or plasma insulin when glucose intake fluctuates. For example, glucose tolerance encompasses the ability to reduce the level of plasma glucose back to a level before the intake of glucose.

The term “subject” refers to a mammal, such as a domestic pet (for example, a dog or cat), or human. Preferably, the subject is a human. In a more preferred embodiment, the human does not suffer from cancer, or has not been diagnosed with cancer. In another embodiment, the human does not suffer from osteoporosis, or has not been diagnosed with osteoporosis. In one embodiment, the subject is a male human. In another embodiment, the subject is a female human (for example, a pre-menopausal woman, or one who is not in need of hormone replacement).

“Therapeutically effective amount” or “pharmaceutically effective amount” refers to the amount which, when administered to a subject or patient for treating a disease, is sufficient to effect such treatment for the disease.

“Treatment” or “treating” includes (1) inhibiting a disease in a subject or patient experiencing or displaying the pathology or symptomatology of the disease (e.g., arresting further development of the pathology and/or symptomatology), (2) ameliorating a disease in a subject or patient that is experiencing or displaying the pathology or symptomatology of the disease (e.g., reversing the pathology and/or symptomatology), and/or (3) effecting any measurable decrease in a disease in a subject or patient that is experiencing or displaying the pathology or symptomatology of the disease.

The term “selective estrogen receptor modulator” (SERM) refers to a compound that has estrogen receptor antagonistic activity in some tissue(s) and estrogen receptor agonistic activity in other tissue(s). For example, the SERM can either directly or through its active metabolite function as an estrogen receptor antagonist (“antiestrogen”) in breast tissue, and provide estrogenic or estrogen-like effect on bone tissue and on serum cholesterol levels (i.e. by reducing serum cholesterol).

### Subject

The subject can be a mammal, such as a primate (e.g., a human or monkey), cow, horse, dog, cat, pig, mouse, rat or guinea pig. In one preferred embodiment, the subject is a human. For example, the subject can be a postmenopausal woman or a man. The human can also be less than 65, 50, or 40 years old (e.g., the human can be from about 20 to about 40 years old, a teenager, or an adolescent). In yet another embodiment, the subject does not suffer from osteoporosis, and/or has not been diagnosed with osteoporosis. In yet another embodiment, the subject does not suffer from cancer.

In some variations of one or more of the above embodiments, the subject also suffers from renal disease, cardiovascular disease, diabetes, autoimmune disease, respiratory disease, neurodegenerative disease, liver disease, infectious disease, or cancer, or has undergone or will undergo organ or tissue transplant.

In some variations of one or more of the above embodiments, the subject does not also suffer from renal disease, cardiovascular disease, diabetes, autoimmune disease, respiratory disease, neurodegenerative disease, liver disease, infectious disease, or cancer, or has not or will not undergo transplant.

In some variations of one or more of the above embodiments, the subject has diabetes (e.g., type II diabetes). In some variations of one or more of the above embodiments, the subject does not have diabetes. In some variations of one or more of the above embodiments, the subject exhibits one or more symptoms of diabetes. In some variations of one or more of the above embodiments, the subject does not exhibit any symptoms of diabetes. In some variations of one or more of the above embodiments, the subject has been identified (or diagnosed) as having diabetes. In some variations of one or more of the above embodiments, the subject has been identified (or diagnosed) as not having diabetes. In some variations of one or more of the above embodiments, the level of a marker of diabetes in the subject has been measured or will be measured.

In some variations of one or more of the above embodiments, the subject has elevated levels of at least one biomarker associated with diabetes, cardiovascular disease, renal disease, fatty liver disease or metabolic syndrome. In some variations of one or more of the above embodiments, the subject does not have elevated levels of at least one biomarker associated with diabetes, cardiovascular disease, renal disease, fatty liver disease or metabolic syndrome. In some variations of one or more of the above embodiments, the subject does not have elevated levels of any biomarker associated with diabetes, cardiovascular disease, renal disease, fatty liver disease or metabolic syndrome. In some embodiments, the biomarker is a marker of insulin resistance, leptin resistance, adiponectin resistance, cardiovascular stress, or renal dysfunction. In some embodiments, the biomarker is a marker of insulin resistance. In some embodiments, the biomarker is fasting glucose or hemoglobin A1c. In some embodiments, the biomarker is a marker of leptin resistance. In some embodiments, the biomarker is a marker of adiponectin resistance. In some embodiments, the biomarker is adiponectin. In some embodiments, the biomarker is a marker of cardiovascular stress. In some embodiments, the biomarker is circulating endothelial cells or C-reactive protein. In some embodiments, the biomarker is circulating endothelial cells. In some embodiments, the biomarker is iNOS-positive circulating endothelial cells. In some embodiments, the biomarker is a marker of renal disease. In some embodiments, the biomarker is serum creatinine. In some embodiments, the biomarker is cystatin C. In some embodiments, the biomarker is uric acid.

In some variations of one or more of the above embodiments, the subject has chronic kidney disease (CKD) or exhibits one or more symptoms of CKD. In some variations of one or more of the above embodiments, the subject does not have chronic kidney disease (CKD). In some variations of one or more of the above embodiments, the subject does not exhibit any symptoms of CKD. In some variations of one or more of the above embodiments, the subject has been identified as having CKD. In some variations of one or more of the above embodiments, the subject has been identified as not having CKD. In some variations of one or more of the above embodiments, the level of a marker of CKD in the subject has been measured or will be measured. In some of the embodiments, the CKD is characterized by a serum creatinine level of 1.3-3.0 mg/DL where the subject is a human female or a serum creatinine level of 1.5-3.0 mg/DL where the subject is a human male. In some embodiments, the CKD is stage 4. In some variations of one or more of the above embodiments, the subject does not have stage 4 chronic kidney disease (CKD).

In some variations of one or more of the above embodiments, the subject has diabetic nephropathy (DN) or exhibits one or more symptoms of DN. In some variations of one or more of the above embodiments, the subject does not have diabetic nephropathy (DN). In some variations of one or more of the above embodiments, the subject does not exhibit any symptoms of DN. In some embodiments, the subject has been identified as having DN. In some embodiments, the subject has been identified as not having DN. In some variations of one or more of the above embodiments, the level of a marker of DN in the subject has been measured or will be measured.

In some variations of one or more of the above embodiments, the level of adiponectin in the blood of the subject has been measured or will be measured.

In some variations of one or more of the above embodiments, the level of Angiotensin II in the subject has been measured or will be measured.

In some variations of one or more of the above embodiments, the subject has insulin resistance or exhibits one or more symptoms of insulin resistance. In some variations of one or more of the above embodiments, the subject does not have insulin resistance. In some variations of one or more of the above embodiments, the subject does not exhibit any symptoms of insulin resistance. In some embodiments, the subject has been identified as having insulin resistance. In some embodiments, the subject has been identified as not having insulin resistance. In some variations of one or more of the above embodiments, the level of a marker of insulin resistance in the subject has been measured or will be measured. In some embodiments, the level of hemoglobin A1c in the subject has been measured or will be measured. In some embodiments, a blood sugar level of the subject has been measured or will be measured. In some embodiments, the administration of the ERM reduces the level of hemoglobin A1c or fasting blood glucose in the subject. In some embodiments, a fasting glucose level of the subject has been measured or will be measured. In some embodiments, the insulin sensitivity of the subject has been measured or will be measured by a hyperinsulinemic euglycemic clamp test. In some embodiments, a glucose disposal rate (GDR) in the subject has been measured or will be measured.

In some variations of one or more of the above embodiments, the subject has glucose intolerance or exhibits one or more symptoms of glucose intolerance. In some variations of one or more of the above embodiments, the subject does not have glucose intolerance. In some variations of one or more of the above embodiments, the subject does not exhibit any symptoms of glucose intolerance. In some embodiments, the subject has been identified as having glucose intolerance. In some embodiments, the subject has been identified as not having glucose intolerance. In some variations of one or more of the above embodiments, the level of a marker of glucose intolerance in the subject has been measured or will be measured. In some embodiments, the level of hemoglobin A1c in the subject has been measured or will be measured. In some embodiments, a blood sugar level of the subject has been measured or will be measured. In some embodiments, the administration of the ERM reduces the level of hemoglobin A1c or fasting blood glucose in the subject. In some embodiments, a fasting glucose level of the subject has been measured or will be measured. In some embodiments, the insulin sensitivity of the subject has been measured or will be measured by a hyperinsulinemic euglycemic clamp test. In some embodiments, a glucose disposal rate (GDR) in the subject has been measured or will be measured.

In some variations of one or more of the above embodiments, the subject has cardiovascular disease (CVD) or exhibits one or more symptoms of CVD. In some variations of one or more of the above embodiments, the subject does not have cardiovascular disease (CVD) or does not exhibit any symptoms of CVD. In some variations of one or more of the above embodiments, the subject does not exhibit any symptoms of CVD. In some variations of one or more of the above embodiments, the subject has been identified as having CVD. In some variations of one or more of the above embodiments, the subject has been identified as not having CVD. In some variations of one or more of the above embodiments, the level of a marker of CVD in the subject has been measured or will be measured.

In some variations of one or more of the above embodiments, the number of circulating endothelial cells (CFCs) in the blood of the subject has been measured or will be measured. In some embodiments, the CECs are iNOS-positive circulating endothelial cells. In some embodiments, the administration of the ERM also reduces the level of circulating endothelial cells in the subject. In some embodiments, the administration of the ERM also reduces the level of hemoglobin A1c or fasting blood glucose in the subject.

In some variations of one or more of the above embodiments, the subject has fatty liver disease (FLD) or exhibits one or more symptoms of FLD. In some variations of one or more of the above embodiments, the subject does not have fatty liver disease (FLD) or does not exhibit any symptoms of FLD. In some variations of one or more of the above embodiments, the subject does not exhibit any symptoms of FLD. In some variations of one or more of the above embodiments, the subject has been identified as having FLD. In some variations of one or more of the above embodiments, the subject has been identified as not having FLD. In some variations of one or more of the above embodiments, the level of a marker of FLD in the subject has been measured or will be measured.

In some variations of one or more of the above embodiments, the subject has been identified as having cancer. In some variations of one or more of the above embodiments, the subject has been identified as not having cancer. In some variations of one or more of the above embodiments, the subject has been identified as having cancer and diabetes. In some variations of one or more of the above embodiments, the subject has been identified as not having cancer and/or not having diabetes.

### Body Weight

The present invention relates to, among other things, methods for reducing body weight and treating or preventing obesity. Obesity is a medical condition in which excess body fat has accumulated to the extent that it may have an adverse effect on health. It is typically defined by body mass index (BMI) and may be further evaluated in terms of fat distribution via the waist-hip ratio and total cardiovascular risk factors. BMI is related to both percentage body fat and total body fat.

BMI is calculated by dividing the subject's mass by the square of his or her height (in metric units: kilograms/meters²). The definitions established by the World Health Organization (WHO) in 1997 and published in 2000 are listed below:

| BMI | Classification |
|---|---|
| <18.5 | underweight |
| 18.5-24.9 | normal weight |
| 25.0-29.9 | overweight |
| 30.0-34.9 | class I obesity |
| 35.0-39.9 | class II obesity |
| ≧40.0 | class III obesity |

Obesity increases the risk of many physical and mental conditions. These comorbidities are most commonly shown in metabolic syndrome, a combination of medical disorders which includes: diabetes mellitus type 2, high blood pressure, high blood cholesterol, and high triglyceride levels.

### Pharmaceutical Formulations and Routes of Administration

The ERM may be formulated as a solid or liquid dosage form. Furthermore, the dosage form may provide immediate, modified, sustained, or delayed release of the ERM. The ERM may be formulated as a hard or soft capsule (e.g., a gelatin capsule), a tablet, a syrup, a suspension, a solid dispersion, a wafer, or an elixir. The hard capsule, soft capsule, tablet or wafer may further comprise a protective coating. The formulated ERM may comprise an agent that delays absorption of the ERM. The formulated ERM may also further comprise an agent that enhances solubility or dispersibility. In some variations, the ERM is dispersed in a liposome, an oil-in-water emulsion or a water-in-oil emulsion. In one embodiment, the ERM is formulated as a liquid solution.

The ERMs may be administered by a variety of routes including orally, transdermally, intraarterially, and by injection (e.g., subcutaneously, intravenously, and intraperitoneally). Oral administration is preferred.

The ERMs may be administered orally in the form of a solid or liquid dosage form. In both, the ERM may be coated in a material to protect it from the action of acids and other natural conditions which may inactivate the ERM. The ERMs may be formulated as aqueous solutions, liquid dispersions, (ingestible) tablets, buccal tablets, troches, capsules, elixirs, suspensions, syrups, and wafers. The oral dosage forms may include excipients known in the art, such as binders, disintegrating agents, flavorants, antioxidants, and preservatives. Liquid dosage forms may include diluents such as saline or an aqueous buffer.

The ERMs may also be administered by injection. ERM formulations suitable for injection may include sterile aqueous solutions (where water soluble) or dispersions, and sterile powders for the extemporaneous preparation of sterile injectable solutions or dispersions. The composition may be sterile and be fluid to the extent that easy syringability exists. It may be stable under the conditions of manufacture and storage and be preserved against the contaminating action of microorganisms such as bacteria and fungi. The carrier can be a solvent or dispersion medium containing, for example, water, ethanol, polyol (such as, glycerol, propylene glycol, and liquid polyethylene glycol), suitable mixtures thereof, and vegetable oils. The proper fluidity can be maintained, for example, by the use of a coating such as lecithin, by the maintenance of the required particle size in the case of dispersion and by the use of surfactants. Prevention of the action of microorganisms can be achieved by various antibacterial and antifungal agents, for example, parabens, chlorobutanol, phenol, and ascorbic acid. In many cases, it will be preferable to include isotonic agents, for example, sugars, sodium chloride, or polyalcohols such as mannitol and sorbitol, in the composition. Prolonged absorption of the injectable compositions can be brought about by including in the composition an agent which delays absorption, for example, aluminum monostearate or gelatin.

Sterile injectable solutions can be prepared by incorporating the therapeutic compound in the required amount in an appropriate solvent with one or a combination of ingredients enumerated above, as required, followed by filtered sterilization. Generally, dispersions are prepared by incorporating the therapeutic compound into a sterile carrier which contains a basic dispersion medium and the required other ingredients from those enumerated above. In the case of sterile powders for the preparation of sterile injectable solutions, the methods of preparation include vacuum drying and freeze-drying which yields a powder of the active ingredient (i.e., the therapeutic compound) plus any additional desired ingredient from a previously sterile-filtered solution thereof.

The actual dosage amount of the ERM administered to a subject may be determined by physical and physiological factors such as age, sex, body weight, severity of condition, the type of disease being treated, previous or concurrent therapeutic interventions, idiopathy of the subject and on the route of administration. These factors may be determined by a skilled artisan. The practitioner responsible for administration will typically determine the concentration of active ingredient(s) in a composition and appropriate dose(s) for the individual subject.

In one embodiment, a human subject is administered (for example, orally) the daily doses of ERM provided in the tables below.

| | Daily Dose | Daily Dose | Daily Dose |
|---|---|---|---|
| ERM | Range 1 | Range 2 | Range 3 |
| Raloxifene | from about 0.5 to | from about 0.5 to | from about 1 to |
| | about 10 mg/kg/day | about 8 mg/kg/day | about 3 mg/kg/day |
| Tamoxifen | from about 0.1 to | from about 0.3 to | from about 0.5 to |
| | about 2 mg/kg/day | about 1 mg/kg/day | about 1 mg/kg/day |
| Diethylstilbestrol | from about 0.05 to | from about 0.1 to | from about 0.2 to |
| | about 1 mg/kg/day | about 0.8 mg/kg/day | about 0.6 mg/kg/day |
| Hexestrol | from about 0.1 to | from about 0.3 to | from about 0.5 to |
| | about 2 mg/kg/day | about 1 mg/kg/day | about 1 mg/kg/day |
| Clomiphene | from about 0.05 to | from about 0.1 to | from about 0.3 to |
| | about 5 mg/kg/day | about 3 mg/kg/day | about 1 mg/kg/day |
| Dienestrol | from about 0.05 to | from about 0.1 to | from about 0.3 to |
| | about 5 mg/kg/day | about 3 mg/kg/day | about 1 mg/kg/day |
| 17β-estradiol | from about 0.05 to | from about 0.1 to | from about 0.3 to |
| | about 5 mg/kg/day | about 3 mg/kg/day | about 1 mg/kg/day |
| Estrone | from about 0.05 to | from about 0.1 to | from about 0.2 to |
| | about 1 mg/kg/day | about 0.8 mg/kg/day | about 0.6 mg/kg/day |
| Lasofoxifene | from about 0.05 to | from about 0.1 to | from about 0.3 to |
| | about 5 mg/kg/day | about 3 mg/kg/day | about 1 mg/kg/day |
| Toremifene | from about 0.05 to | from about 0.1 to | from about 0.3 to |
| | about 5 mg/kg/day | about 3 mg/kg/day | about 1 mg/kg/day |

Oral dosage forms of the ERM may provide immediate release or modified release (e.g., sustained or delayed release) of the ERM. In one preferred embodiment, the dosage form provides release of the ERM so that therapeutic plasma levels of the ERM are sustained over 24 hours.

Single or multiple doses of the ERMs are contemplated. Desired time intervals for delivery of multiple doses can be determined by one of ordinary skill in the art employing no more than routine experimentation. As an example, subjects may be administered two doses daily at approximately 12 hour intervals. In some embodiments, the ERM is administered once a day.

The ERMs may be administered on a routine schedule. As used herein a routine schedule refers to a predetermined designated period of time. The routine schedule may encompass periods of time which are identical or which differ in length, as long as the schedule is predetermined. For instance, the routine schedule may involve administration twice a day, every day, every two days, every three days, every four days, every five days, every six days, a weekly basis, a monthly basis or any set number of days or weeks there-between. Alternatively, the predetermined routine schedule may involve administration on a twice daily basis for the first week, followed by a daily basis for several months, etc. In other embodiments, the invention provides that the agent(s) may taken orally and that the timing of which is or is not dependent upon food intake. Thus, for example, the agent can be taken every morning and/or every evening, regardless of when the subject has eaten or will eat.

### Combination Therapy

In addition to being used as a monotherapy, the ERMs may also find use in combination therapies. Effective combination therapy may be achieved with a single composition or pharmacological formulation that includes both agents, or with two distinct compositions or formulations, administered at the same time, wherein one composition includes a compound of this invention, and the other includes the second agent(s). Alternatively, the therapy may precede or follow the other agent treatment by intervals ranging from minutes to months.

Various combinations may be employed, such as when a ERM is “A” and “B” represents a secondary agent, non-limiting examples of which are described below:

A/B/A B/A/B B/B/A A/A/B A/B/B B/A/A A/B/B/B B/A/B/B
B/B/B/A B/B/A/B A/A/B/B A/B/A/B A/B/B/A B/B/A/A B/A/B/A
B/A/A/B A/A/A/B B/A/A/A A/B/A/A A/A/B/A

The additional agent or agents may be selected from weight loss agents and anti-obesity agents. The agents may be selected from substances showing appetite-suppressing or energy metabolism-boosting activity, lipid degradation-suppressing activity, retardation activity of gastric emptying, protein tyrosine phosphatase (PTP) 1b-inhibiting activity and DPP IV-inhibiting activity. Examples of suitable agents include, but are not limited to, PYY (peptide YY), cholecytokinin (CCK), oxyntomodulin, ghrelin antagonist, NPY antagonist, cannabinoid CB1 receptor antagonist, a lipase inhibitor, a monoamine reuptake inhibitor, an anticonvulsant, a glucose sensitizer, a incretin mimetic, an amylin analog, a GLP-1 analog, a Y receptor peptide, a 5-HT2C serotonin receptor agonist, an opioid receptor antagonist, an appetite suppressant, an anorectic, a hormone, or a pharmaceutically acceptable salt thereof.

The agent may be a cannabinoid CB1 receptor antagonist. Examples of cannabinoid CB1 receptor antagonists include, but are not limited to, rimonabant, surinabant, and pharmaceutically acceptable salts thereof.

The agent may be a lipase inhibitor. Examples of lipase inhibitors include, but are not limited to, orlistat, cetilistat, and pharmaceutically acceptable salts thereof.

The agent may be a monoamine reuptake inhibitor, such as an SSRI or an SNRI. Examples of monoamine reuptake inhibitors include sibutramine, bupropion, citalopram, escitalopram, fluoxetine, paroxetine, sertraline, duloxetine, milnacipran, mirtazapine, venlafaxine, desvenlafaxine, and pharmaceutically acceptable salts thereof.

The agent may be a serotoning-noradrenaline-dopamine reuptake inhibitor, such as tesofensine or a pharmaceutically acceptable salt thereof.

The agent may be an anticonvulsant. Examples of anticonvulsants include, but are not limited to, topiramate, zonisamide, and pharmaceutically acceptable salts thereof.

The agent may be a glucose sensitizer. Examples of glucose sensitizers include, but are not limited to, metformin and pharmaceutically acceptable salts thereof.

The agent may be an incretin mimetic. Examples of incretin mimetics include, but are not limited to, exenatide and pharmaceutically acceptable salts thereof.

The agent may be an amylin or an analog thereof. Examples of amylin analogs include, but are not limited to, pramlintide and pharmaceutically acceptable salts thereof.

The agent may be a GLP-1 or an analog thereof. Examples of GLP-1 analogs include, but are not limited to, liraglutide and pharmaceutically acceptable salts thereof.

The agent may be a Y receptor peptide. Examples of Y receptor peptides include, but are not limited to, obinepitide and pharmaceutically acceptable salts thereof.

The agent may be a 5-HT_{2C} serotonin receptor agonist. Examples of 5-HT_{2C} serotonin receptor agonists include, but are not limited to, lorcaserin and pharmaceutically acceptable salts thereof.

The agent may be an opioid receptor antagonist. Examples of opioid receptor antagonists include, but are not limited to, naltrexone and pharmaceutically acceptable salts thereof.

The agent may be an appetite suppressant. Examples of appetite suppressants include, but are not limited to, phentermine, diethylpropion and pharmaceutically acceptable salts thereof.

The agent may be an anorectic. Examples of an anorectic include, but are not limited to, phendimetrazine and pharmaceutically acceptable salts thereof.

The agent may be a hormone. Examples of hormones include, but are not limited to, insulin, leptin, and pharmaceutically acceptable salts thereof.

In one embodiment, the ERM is co-administered with one or more agents selected from rimonabant, surinabant, orlistat, cetilistat, sibutramine, bupropion, citalopram, escitalopram, fluoxetine, paroxetine, sertraline, duloxetine, milnacipran, mirtazapine, venlafaxine, desvenlafaxine, tesofensine, topiramate, zonisamide, metformin, exenatide, pramlintide, liraglutide, obinepitide, lorcaserin, naltrexone, phentermine, phendimetrazine, insulin, leptin, and pharmaceutically acceptable salts thereof.

In another embodiment, the ERM is co-administered with one or more agents selected from phentermine, diethyipropion, phendimetrazine, orlistat, bupropion, topiramate, zonisamide, metformin, and pharmaceutically acceptable salts thereof.

In yet another embodiment, the ERM is co-administered with a lipase inhibitor. For example, the ERM may be co-administered with orlistat or a pharmaceutically acceptable salt thereof. In one preferred embodiment, raloxifene or a pharmaceutically acceptable salt thereof is co-administered with orlistat or a pharmaceutically acceptable salt thereof. In another preferred embodiment, tamoxifen or a pharmaceutically acceptable salt thereof is co-administered with orlistat or a pharmaceutically acceptable salt thereof. In yet another preferred embodiment, diethylstilbestrol or a pharmaceutically acceptable salt thereof is co-administered with orlistat or a pharmaceutically acceptable salt thereof. In yet another preferred embodiment, hexestrol or a pharmaceutically acceptable salt thereof is co-administered with orlistat or a pharmaceutically acceptable salt thereof. In yet another preferred embodiment, clomiphene or a pharmaceutically acceptable salt thereof is co-administered with orlistat or a pharmaceutically acceptable salt thereof.

Examples of combination therapies where an ERM is combined with a second agent are provided in the table below. The individual agents in these combinations may be administered together in a single oral dosage form, co-administered at the same time (simultaneously) or near the same time in separate oral dosage forms, or at different times during the same day. For example, raloxifene (or a pharmaceutically acceptable salt thereof) may be administered once daily and orlistat may be administered three times a day with each main meal containing fat (during or up to 1 hour after the meal).

| ERM/ | | Second Agent |
|---|---|---|
| ERM Daily Dose | Second Agent | Daily Dose |
| Raloxifene | Orlistat | from about 120 to about 600 mg (e.g., |
| (e.g., raloxifene | | about 120 mg three times a day with |
| hydrochloride) | | each main meal containing fat (during |
| from about 10 to about | | or up to 1 hour after the meal)) |
| 200 mg | Phentermine | from about 8 to about 50 mg (e.g., from |
| (e.g., about 60 mg | (e.g., phentermine | about 15 to about 37.5 mg) |
| once daily) | hydrochloride) | |
| | Diethylpropion | from about 25 to about 225 mg (e.g., |
| | (e.g., | from about 50 to about 100 mg, such as |
| | diethylpropion | 75 mg once daily in an extended |
| | hydrochloride) | release formulation or 25 mg three |
| | | times daily, one hour before meals, and |
| | | in midevening if desired to overcome |
| | | night hunger) |
| | Phendimetrazine | from about 50 to about 200 mg (e.g., as |
| | (e.g., | an extended release once-daily dosage |
| | phendimetrazine | form containing about 105 mg) |
| | tartrate) | |
| | Bupropion | from about 150 to about 600 mg (e.g., |
| | (e.g., bupropion | 300 mg daily, such as by a 150 mg |
| | hydrochloride or | extended release dosage form twice |
| | bupropion | daily, or alternatively as a 300 mg |
| | hydrobromide) | extended release dosage form once |
| | | daily) |
| | Topiramate | from about 25 to about 600 mg (e.g., |
| | | from about 100 to about 500 mg, such |
| | | as 200, 250, 300, 350, or 400 mg) |
| | Zonisamide | from about 10 to about 400 mg (e.g., |
| | | 25 to about 200 mg, such as 25, 50, 75, |
| | | 100, 125, 150, 175, or 200 mg) |
| | Metformin | from about 500 mg to about 3000 mg |
| | (e.g., metformin | (e.g., from about 500 to about 2550 mg |
| | hydrochloride) | or about 2000 mg) |
| Tamoxifen | Orlistat | from about 120 to about 600 mg (e.g., |
| (e.g., tamoxifen | | about 120 mg three times a day with |
| citrate) | | each main meal containing fat (during |
| from about 5 to about | | or up to 1 hour after the meal)) |
| 80 mg (e.g., from | Phentermine | from about 8 to about 50 mg (e.g., from |
| about 20 to about 40 | (e.g., phentermine | about 15 to about 37.5 mg) |
| mg, such as 20 mg) | hydrochloride) | |
| | Diethylpropion | from about 25 to about 225 mg (e.g., |
| | (e.g., | from about 50 to about 100 mg, such as |
| | diethylpropion | 75 mg once daily in an extended |
| | hydrochloride) | release formulation or 25 mg three |
| | | times daily, one hour before meals, and |
| | | in midevening if desired to overcome |
| | | night hunger) |
| | Phendimetrazine | from about 50 to about 200 mg (e.g., as |
| | (e.g., | an extended release once-daily dosage |
| | phendimetrazine | form containing about 105 mg) |
| | tartrate) | |
| | Bupropion | from about 150 to about 600 mg (e.g., |
| | (e.g., bupropion | 300 mg daily, such as by a 150 mg |
| | hydrochloride or | extended release dosage form twice |
| | bupropion | daily, or alternatively as a 300 mg |
| | hydrobromide) | extended release dosage form once |
| | | daily) |
| | Topiramate | from about 25 to about 600 mg (e.g., |
| | | from about 100 to about 500 mg, such |
| | | as 200, 250, 300, 350, or 400 mg) |
| | Zonisamide | from about 10 to about 400 mg (e.g., |
| | | 25 to about 200 mg, such as 25, 50, 75, |
| | | 100, 125, 150, 175, or 200 mg) |
| | Metformin | from about 500 mg to about 3000 mg |
| | (e.g., metformin | (e.g., from about 500 to about 2550 mg |
| | hydrochloride) | or about 2000 mg) |
| Diethylstilbestrol | Orlistat | from about 120 to about 600 mg (e.g., |
| from about 0.1 to | | about 120 mg three times a day with |
| about 20 mg, e.g., | | each main meal containing fat (during |
| from about 0.1 to | | or up to 1 hour after the meal)) |
| about 5 mg | Phentermine | from about 8 to about 50 mg (e.g., from |
| | (e.g., phentermine | about 15 to about 37.5 mg) |
| | hydrochloride) | |
| | Diethylpropion | from about 25 to about 225 mg (e.g., |
| | (e.g., | from about 50 to about 100 mg, such as |
| | diethylpropion | 75 mg once daily in an extended |
| | hydrochloride) | release formulation or 25 mg three |
| | | times daily, one hour before meals, and |
| | | in midevening if desired to overcome |
| | | night hunger) |
| | Phendimetrazine | from about 50 to about 200 mg (e.g., as |
| | (e.g., | an extended release once-daily dosage |
| | phendimetrazine | form containing about 105 mg) |
| | tartrate) | |
| | Bupropion | from about 150 to about 600 mg (e.g., |
| | (e.g., bupropion | 300 mg daily, such as by a 150 mg |
| | hydrochloride or | extended release dosage form twice |
| | bupropion | daily, or alternatively as a 300 mg |
| | hydrobromide) | extended release dosage form once |
| | | daily) |
| | Topiramate | from about 25 to about 600 mg (e.g., |
| | | from about 100 to about 500 mg, such |
| | | as 200, 250, 300, 350, or 400 mg) |
| | Zonisamide | from about 10 to about 400 mg (e.g., |
| | | 25 to about 200 mg, such as 25, 50, 75, |
| | | 100, 125, 150, 175, or 200 mg) |
| | Metformin | from about 500 mg to about 3000 mg |
| | (e.g., metformin | (e.g., from about 500 to about 2550 mg |
| | hydrochloride) | or about 2000 mg) |
| Hexestrol | Orlistat | from about 120 to about 600 mg (e.g., |
| from about 0.1 to | | about 120 mg three times a day with |
| about 20 mg, e.g., | | each main meal containing fat (during |
| from about 0.1 to | | or up to 1 hour after the meal)) |
| about 5 mg | Phentermine | from about 8 to about 50 mg (e.g., from |
| | (e.g., phentermine | about 15 to about 37.5 mg) |
| | hydrochloride) | |
| | Diethylpropion | from about 25 to about 225 mg (e.g., |
| | (e.g., | from about 50 to about 100 mg, such as |
| | diethylpropion | 75 mg once daily in an extended |
| | hydrochloride) | release formulation or 25 mg three |
| | | times daily, one hour before meals, and |
| | | in midevening if desired to overcome |
| | | night hunger) |
| | Phendimetrazine | from about 50 to about 200 mg (e.g., as |
| | (e.g., | an extended release once-daily dosage |
| | phendimetrazine | form containing about 105 mg) |
| | tartrate) | |
| | Bupropion | from about 150 to about 600 mg (e.g., |
| | (e.g., bupropion | 300 mg daily, such as by a 150 mg |
| | hydrochloride or | extended release dosage form twice |
| | bupropion | daily, or alternatively as a 300 mg |
| | hydrobromide) | extended release dosage form once |
| | | daily) |
| | Topiramate | from about 25 to about 600 mg (e.g., |
| | | from about 100 to about 500 mg, such |
| | | as 200, 250, 300, 350, or 400 mg) |
| | Zonisamide | from about 10 to about 400 mg (e.g., |
| | | 25 to about 200 mg, such as 25, 50, 75, |
| | | 100, 125, 150, 175, or 200 mg) |
| | Metformin | from about 500 mg to about 3000 mg |
| | (e.g., metformin | (e.g., from about 500 to about 2550 mg |
| | hydrochloride) | or about 2000 mg) |
| Clomiphene | Orlistat | from about 120 to about 600 mg (e.g., |
| (e.g., clomiphene | | about 120 mg three times a day with |
| citrate) | | each main meal containing fat (during |
| from about 10 or 20 | | or up to 1 hour after the meal)) |
| mg to about 150 mg, | Phentermine | from about 8 to about 50 mg (e.g., from |
| e.g. from about 50 to | (e.g., phentermine | about 15 to about 37.5 mg) |
| about 100 mg | hydrochloride) | |
| | Diethylpropion | from about 25 to about 225 mg (e.g., |
| | (e.g., | from about 50 to about 100 mg, such as |
| | diethylpropion | 75 mg once daily in an extended |
| | hydrochloride) | release formulation or 25 mg three |
| | | times daily, one hour before meals, and |
| | | in midevening if desired to overcome |
| | | night hunger) |
| | Phendimetrazine | from about 50 to about 200 mg (e.g., as |
| | (e.g., | an extended release once-daily dosage |
| | phendimetrazine | form containing about 105 mg) |
| | tartrate) | |
| | Bupropion | from about 150 to about 600 mg (e.g., |
| | (e.g., bupropion | 300 mg daily, such as by a 150 mg |
| | hydrochloride or | extended release dosage form twice |
| | bupropion | daily, or alternatively as a 300 mg |
| | hydrobromide) | extended release dosage form once |
| | | daily) |
| | Topiramate | from about 25 to about 600 mg (e.g., |
| | | from about 100 to about 500 mg, such |
| | | as 200, 250, 300, 350, or 400 mg) |
| | Zonisamide | from about 10 to about 400 mg (e.g., |
| | | 25 to about 200 mg, such as 25, 50, 75, |
| | | 100, 125, 150, 175, or 200 mg) |
| | Metformin | from about 500 mg to about 3000 mg |
| | (e.g., metformin | (e.g., from about 500 to about 2550 mg |
| | hydrochloride) | or about 2000 mg) |
| Dienestrol | Orlistat | from about 120 to about 600 mg (e.g., |
| from about 0.1 to | | about 120 mg three times a day with |
| about 20 mg, e.g., | | each main meal containing fat (during |
| from about 0.1 to | | or up to 1 hour after the meal)) |
| about 5 mg | Phentermine | from about 8 to about 50 mg (e.g., from |
| | (e.g., phentermine | about 15 to about 37.5 mg) |
| | hydrochloride) | |
| | Diethylpropion | from about 25 to about 225 mg (e.g., |
| | (e.g., | from about 50 to about 100 mg, such as |
| | diethylpropion | 75 mg once daily in an extended |
| | hydrochloride) | release formulation or 25 mg three |
| | | times daily, one hour before meals, and |
| | | in midevening if desired to overcome |
| | | night hunger) |
| | Phendimetrazine | from about 50 to about 200 mg (e.g., as |
| | (e.g., | an extended release once-daily dosage |
| | phendimetrazine | form containing about 105 mg) |
| | tartrate) | |
| | Bupropion | from about 150 to about 600 mg (e.g., |
| | (e.g., bupropion | 300 mg daily, such as by a 150 mg |
| | hydrochloride or | extended release dosage form twice |
| | bupropion | daily, or alternatively as a 300 mg |
| | hydrobromide) | extended release dosage form once |
| | | daily) |
| | Topiramate | from about 25 to about 600 mg (e.g., |
| | | from about 100 to about 500 mg, such |
| | | as 200, 250, 300, 350, or 400 mg) |
| | Zonisamide | from about 10 to about 400 mg (e.g., |
| | | 25 to about 200 mg, such as 25, 50, 75, |
| | | 100, 125, 150, 175, or 200 mg) |
| | Metformin | from about 500 mg to about 3000 mg |
| | (e.g., metformin | (e.g., from about 500 to about 2550 mg |
| | hydrochloride) | or about 2000 mg) |
| Lasofoxifene | Orlistat | from about 120 to about 600 mg (e.g., |
| from about 10 to about | | about 120 mg three times a day with |
| 200 mg | | each main meal containing fat (during |
| (e.g., about 60 mg | | or up to 1 hour after the meal)) |
| once daily) | Phentermine | from about 8 to about 50 mg (e.g., from |
| | (e.g., phentermine | about 15 to about 37.5 mg) |
| | hydrochloride) | |
| | Diethylpropion | from about 25 to about 225 mg (e.g., |
| | (e.g., | from about 50 to about 100 mg, such as |
| | diethylpropion | 75 mg once daily in an extended |
| | hydrochloride) | release formulation or 25 mg three |
| | | times daily, one hour before meals, and |
| | | in midevening if desired to overcome |
| | | night hunger) |
| | Phendimetrazine | from about 50 to about 200 mg (e.g., as |
| | (e.g., | an extended release once-daily dosage |
| | phendimetrazine | form containing about 105 mg) |
| | tartrate) | |
| | Bupropion | from about 150 to about 600 mg (e.g., |
| | (e.g., bupropion | 300 mg daily, such as by a 150 mg |
| | hydrochloride or | extended release dosage form twice |
| | bupropion | daily, or alternatively as a 300 mg |
| | hydrobromide) | extended release dosage form once |
| | | daily) |
| | Topiramate | from about 25 to about 600 mg (e.g., |
| | | from about 100 to about 500 mg, such |
| | | as 200, 250, 300, 350, or 400 mg) |
| | Zonisamide | from about 10 to about 400 mg (e.g., |
| | | 25 to about 200 mg, such as 25, 50, 75, |
| | | 100, 125, 150, 175, or 200 mg) |
| | Metformin | from about 500 mg to about 3000 mg |
| | (e.g., metformin | (e.g., from about 500 to about 2550 mg |
| | hydrochloride) | or about 2000 mg) |
| Toremifene | Orlistat | from about 120 to about 600 mg (e.g., |
| from about 10 to about | | about 120 mg three times a day with |
| 200 mg | | each main meal containing fat (during |
| (e.g., about 60 mg | | or up to 1 hour after the meal)) |
| once daily) | Phentermine | from about 8 to about 50 mg (e.g., from |
| | (e.g., phentermine | about 15 to about 37.5 mg) |
| | hydrochloride) | |
| | Diethylpropion | from about 25 to about 225 mg (e.g., |
| | (e.g., | from about 50 to about 100 mg, such as |
| | diethylpropion | 75 mg once daily in an extended |
| | hydrochloride) | release formulation or 25 mg three |
| | | times daily, one hour before meals, and |
| | | in midevening if desired to overcome |
| | | night hunger) |
| | Phendimetrazine | from about 50 to about 200 mg (e.g., as |
| | (e.g., | an extended release once-daily dosage |
| | phendimetrazine | form containing about 105 mg) |
| | tartrate) | |
| | Bupropion | from about 150 to about 600 mg (e.g., |
| | (e.g., bupropion | 300 mg daily, such as by a 150 mg |
| | hydrochloride or | extended release dosage form twice |
| | bupropion | daily, or alternatively as a 300 mg |
| | hydrobromide) | extended release dosage form once |
| | | daily) |
| | Topiramate | from about 25 to about 600 mg (e.g., |
| | | from about 100 to about 500 mg, such |
| | | as 200, 250, 300, 350, or 400 mg) |
| | Zonisamide | from about 10 to about 400 mg (e.g., |
| | | 25 to about 200 mg, such as 25, 50, 75, |
| | | 100, 125, 150, 175, or 200 mg) |
| | Metformin | from about 500 mg to about 3000 mg |
| | (e.g., metformin | (e.g., from about 500 to about 2550 mg |
| | hydrochloride) | or about 2000 mg) |

It is contemplated that other anti-obesity agents may be used in conjunction with the treatments of the current invention. For example, for dogs, the anti-obesity agent dirlotapide may be co-administered with the ERM.

### EXAMPLES

### Example 1

Male C57BL6/J mice were tested as follows. The animal room was lighted entirely with artificial fluorescent lighting on controlled 12 hr light/dark cycle (6 a.m. to 6 p.m. light). The normal temperature and relative humidity ranges in the animal rooms were maintained at 22±4° C. and 50±15%, respectively. The animal room was set for 15 air exchanges per hour. Filtered tap water acidified to a pH of 2.5 to 3.0 was provided ad libitum. High fat diet was provided ad libitum.

After a 1 week acclimation, the mice were grouped into cohorts of 6 mice each. 6 DIO (diet induced obesity) and 6 lean C57BL6/J mice were used as naïve controls. Each drug was administered via drinking water for 8 weeks. The drug containing drinking water solutions were made fresh weekly. Water intake, food intake, and body weights were recorded weekly.

This experiment was run in two phases, Phase 1 (prevention of weight gain in lean mice) and Phase 2 (weight loss in obese mice). In the phase 1 experiment, the mice were placed on high fat diet at the same time administration of the drug was initiated. In the phase 2 experiment, drug treatment was initiated on 18 week old mice, which were previously on a high fat diet for approximately 12 weeks. In addition to the measurements noted above, a single DEXA Scan analysis was ran on the mice in Phase 2. After treatment, mice underwent body tissue composition analysis by Echo MRI (NMR) to measure their fat and lean mass content.

Body weight change was calculated for each mouse by subtracting the body weight on the first day of dosing (baseline) from the body weight on the last day of dosing (final) and calculating the percent change from baseline body weight: [=100*(Final Weight−BaselineWeight)/BaselineWeight]. Average percent body weight change was then calculated for each treatment group. Error bars represent standard error of the mean (SEM).

Food intake per mouse per day was calculated based on food intake per cage. Mean food intake for each treatment group was then calculated weighted by the number of mice per cage. Similarly, water intake per mouse per day was calculated based on water intake per cage. Mean water intake for each treatment group was then calculated weighted by the number of mice per cage.

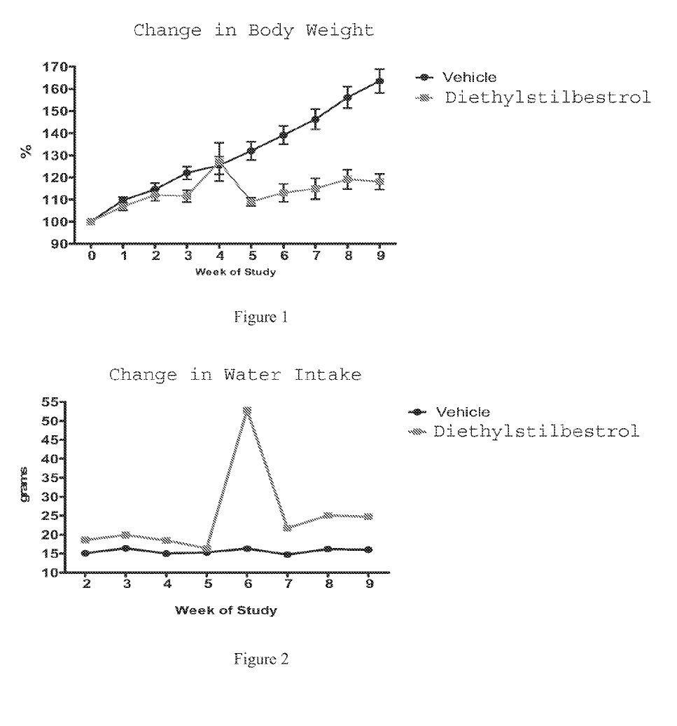

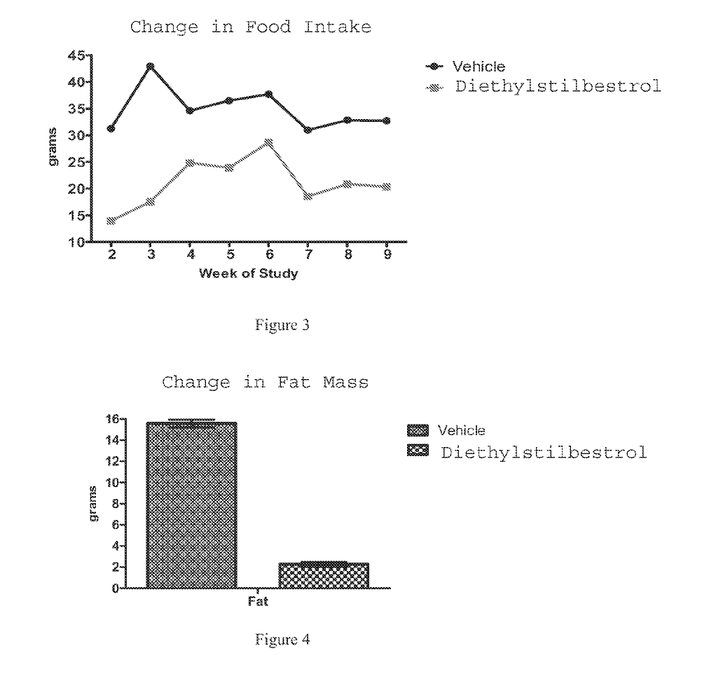

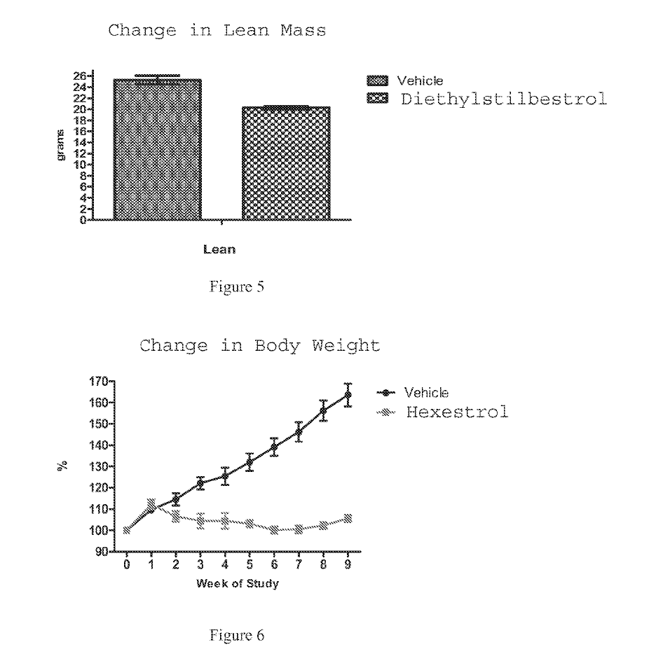

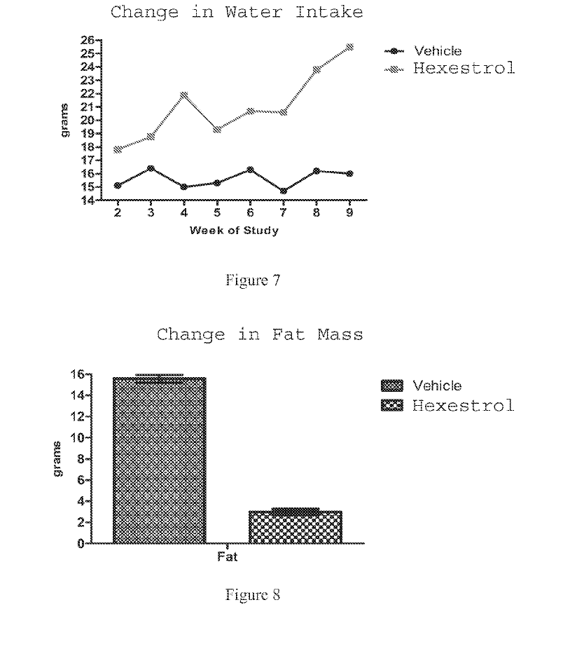

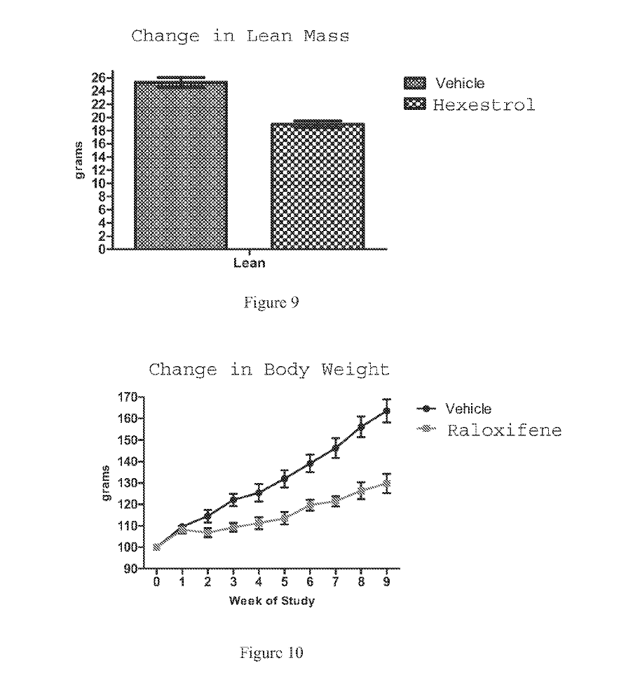

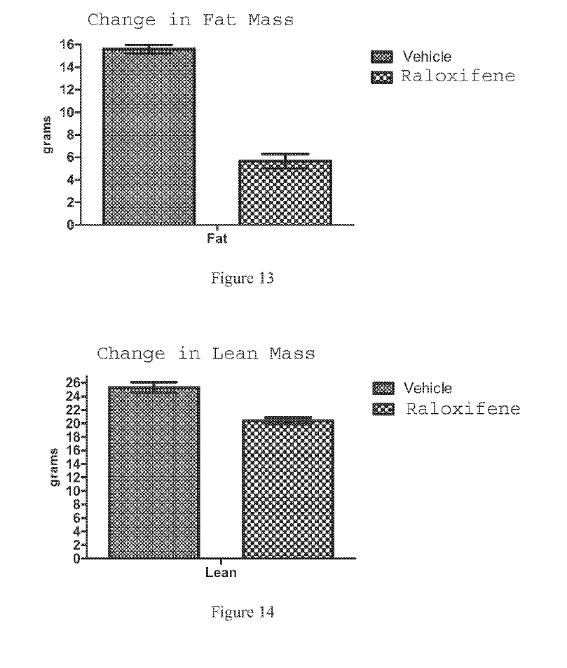

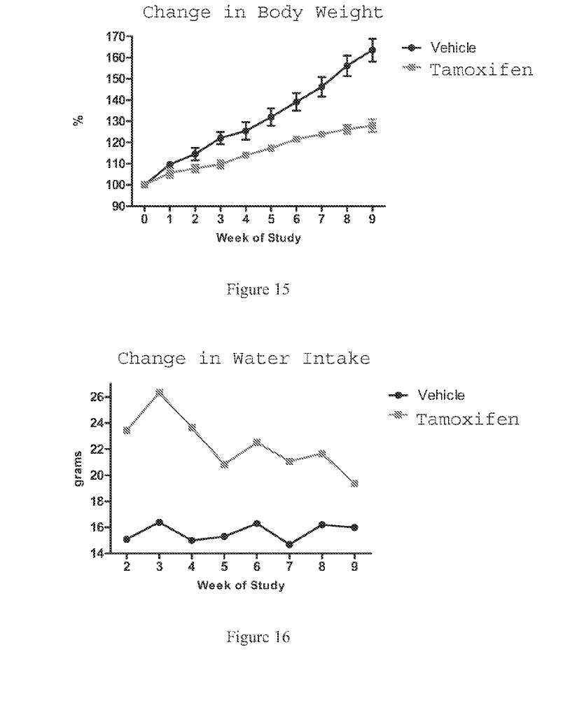

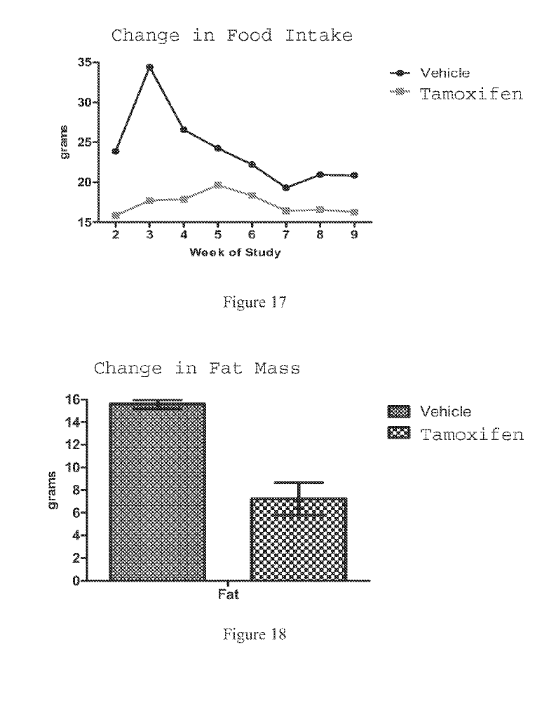

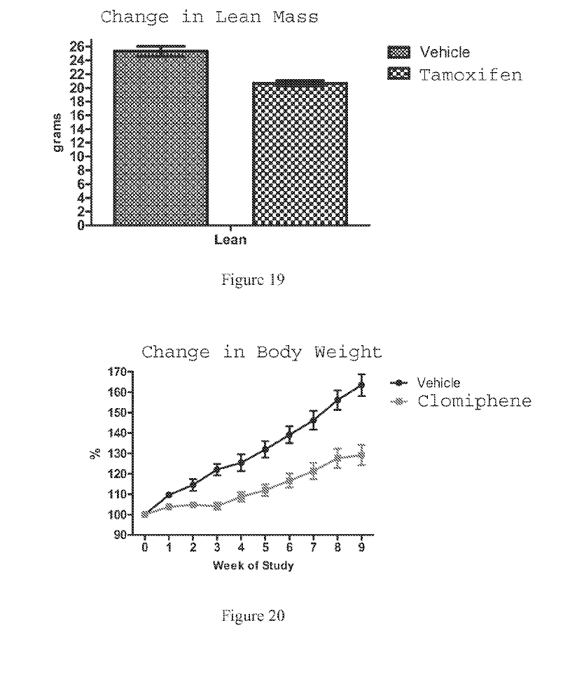

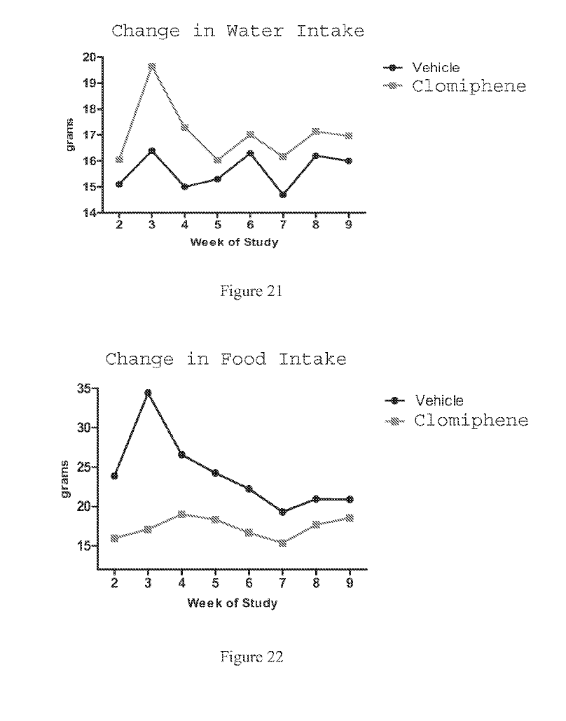

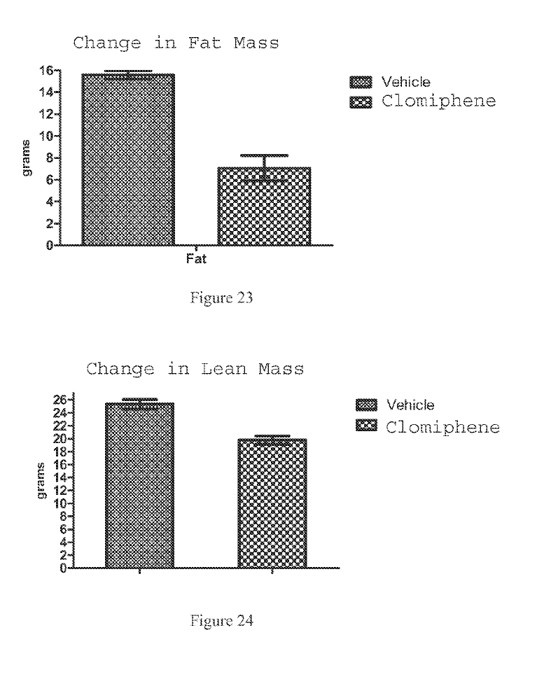

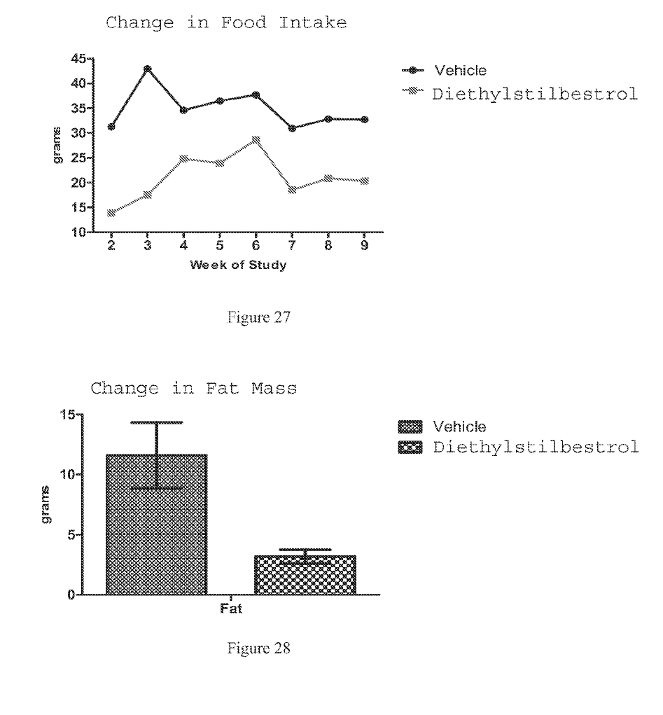

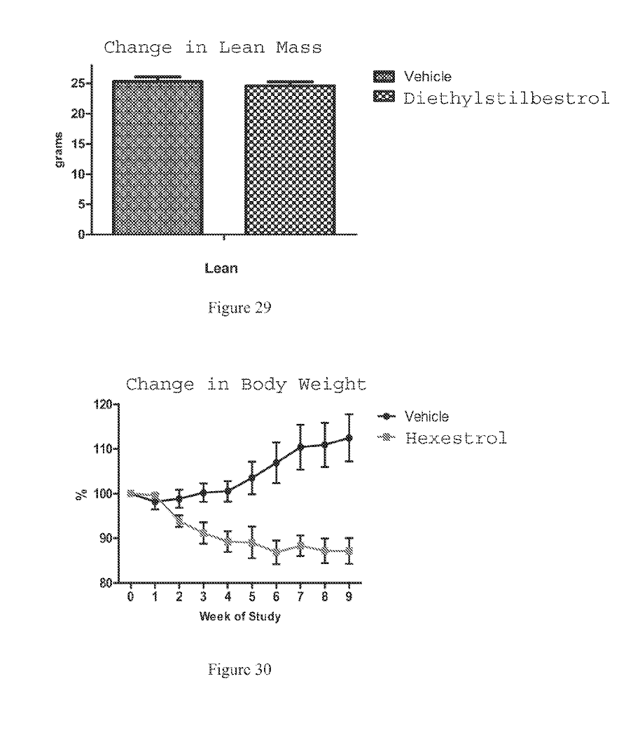

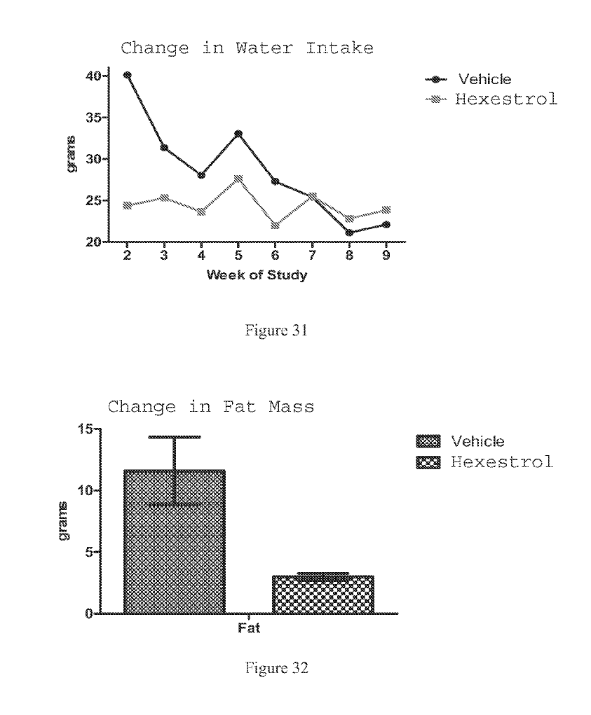

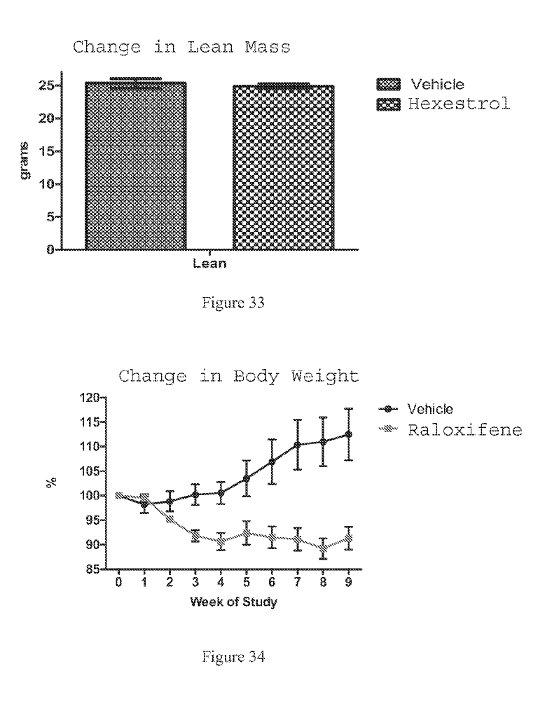

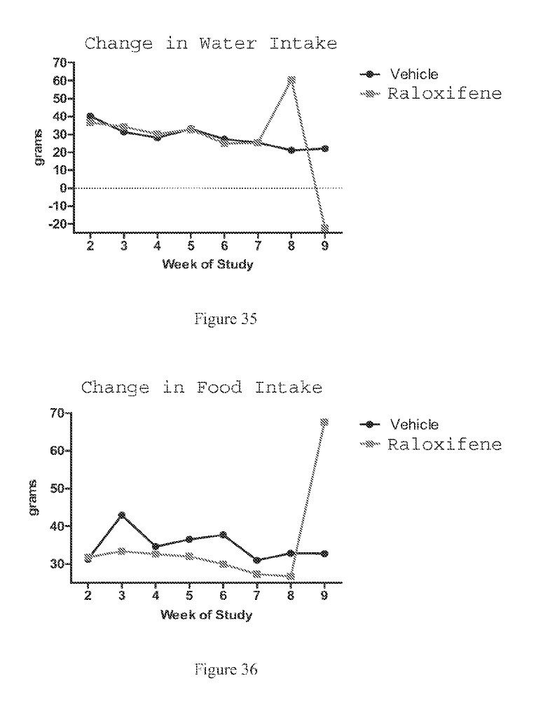

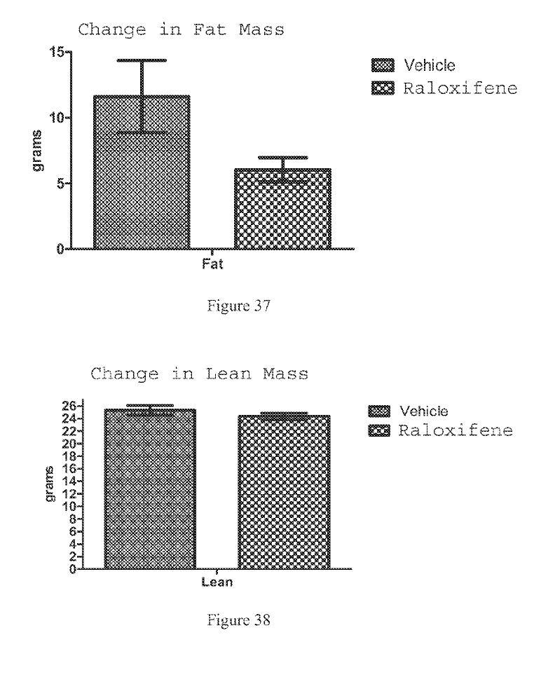

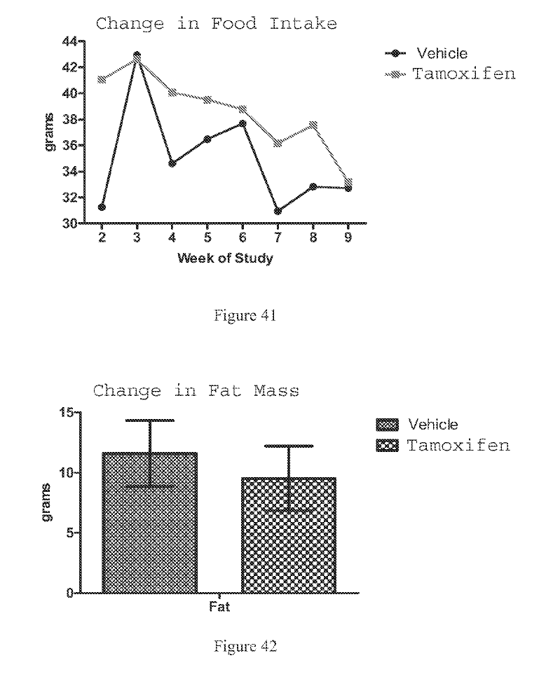

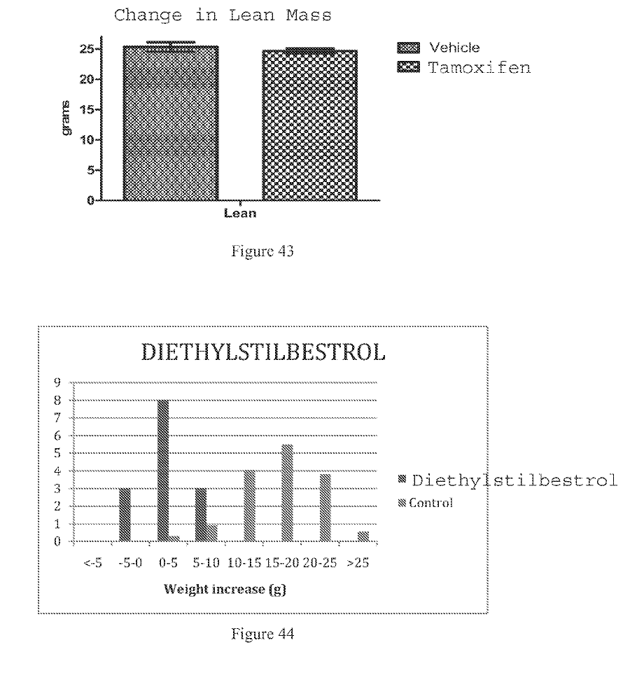

The results for diethylstilbestrol, raloxifene, tamoxifen, clomiphene, and hexestrol are shown in FIG. 1-43. Error bars represent standard error of the mean (SEM).

In both phase 1 and 2 studies, the drugs were administered through the water given to the mice. The concentration of each drug in the water is provided below:
Diethylstilbestrol: 2 mg/500 mL (1 mg/kg/day)
Raloxifene: 20 mg/500 mL (10 mg/kg/day)
Tamoxifen: 4 mg/500 mL (2 mg/kg/day)
Clomiphene: 10 mg/500 mL (5 mg/kg/day)
Hexestrol: 4 mg/500 mL (2 mg/kg/day)
   A mouse typically drinks about 5 mL of water a day.

### Phase 1 (Prevention of Weight Gain in Lean Mice)

The change in body weight, water intake, food intake, fat, and lean mass for mice treated with diethylstilbestrol are shown in FIGS. 1-5, respectively.

The change in body weight, water intake, fat, and lean mass for mice treated with hexestrol in FIGS. 6-9, respectively.

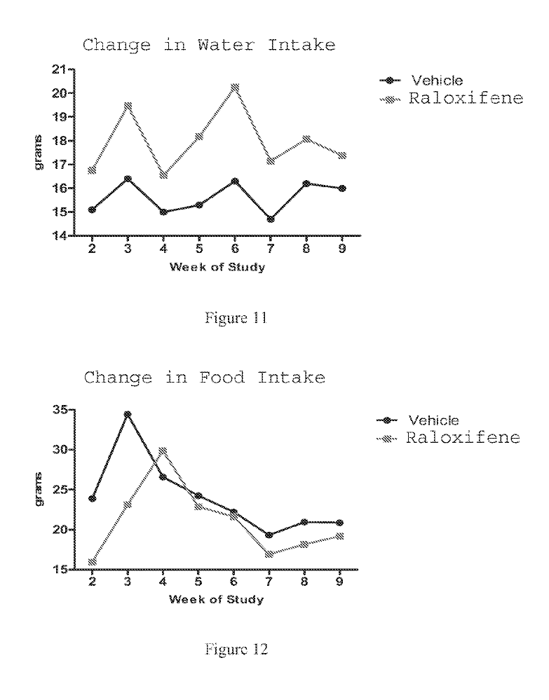

The change in body weight, water intake, food intake, fat, and lean mass for mice treated with raloxifene are shown in FIGS. 10-14, respectively.

The change in body weight, water intake, food intake, fat, and lean mass for mice treated with tamoxifen are shown in FIGS. 15-19, respectively.

The change in body weight, water intake, food intake, fat, and lean mass for mice treated with clomiphene are shown in FIGS. 20-24, respectively.

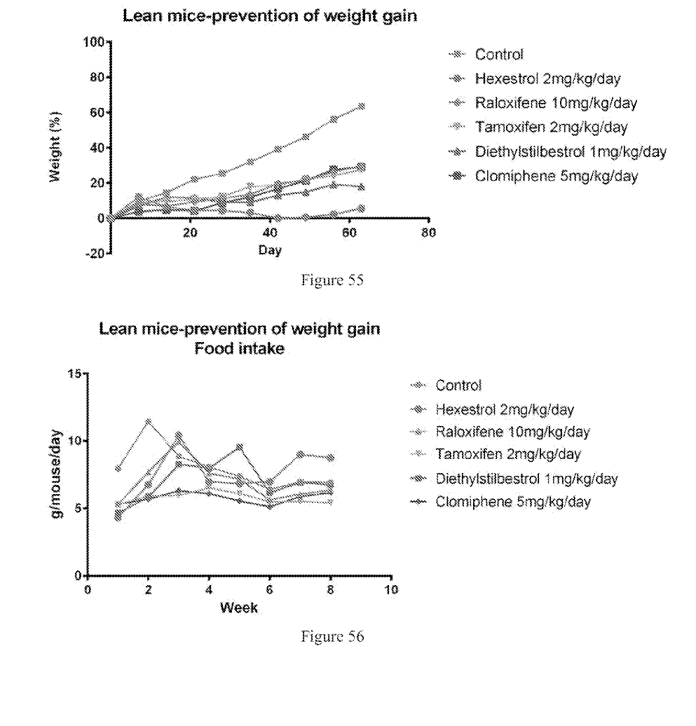

The change in body weight over time in treated and control mice is shown in FIGS. 54 and 55. All mice treated with the estrogen modulating compounds (hexestrol, raloxifene, tamoxifen, diethylstilbestrol, and clomiphene) exhibited a significant decrease in lean mouse body weight accumulation over the 8 week period, with hexestrol being most effective with 35.0±11.3% reduction from control mice.

The change in food intake over time in treated and control mice is shown in FIG. 56. Food intake did not significantly change in any of the treatment groups in comparison to control mice.

FIG. 57 is a bar graph showing the change in fat and lean mass in treated and control mice after the 8-week study as measured by a NMR body composition analysis. All treated mice exhibited a significant reduction in fat tissue mass in comparison to control mice. Lean tissue mass remained unchanged from control mice in all treated groups.

### Phase 2 (Weight Loss in Obese Mice)

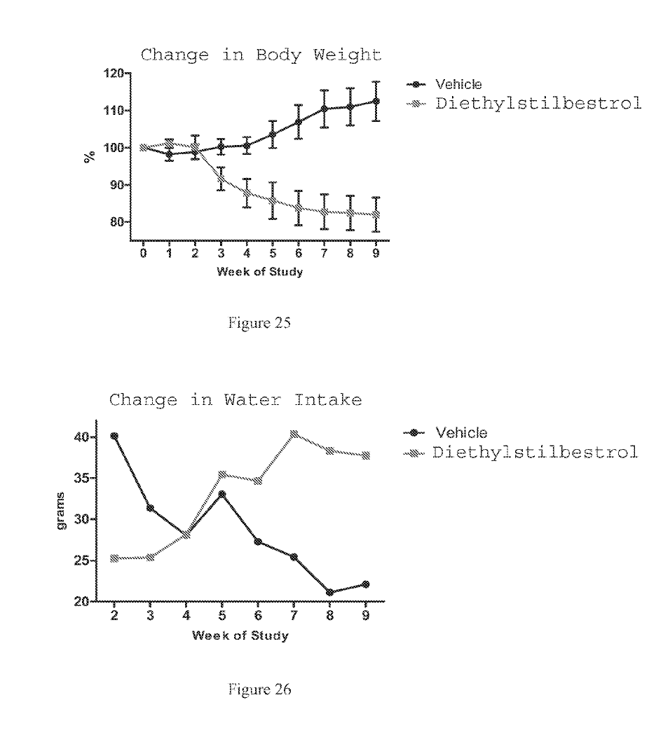

The change in body weight, water intake, food intake, fat, and lean mass for mice treated with diethylstilbestrol are shown in FIGS. 25-29, respectively.

The change in body weight, water intake, fat, and lean mass for mice treated with hexestrol in FIGS. 30-33, respectively.

The change in body weight, water intake, food intake, fat, and lean mass for mice treated with raloxifene are shown in FIGS. 34-38, respectively.

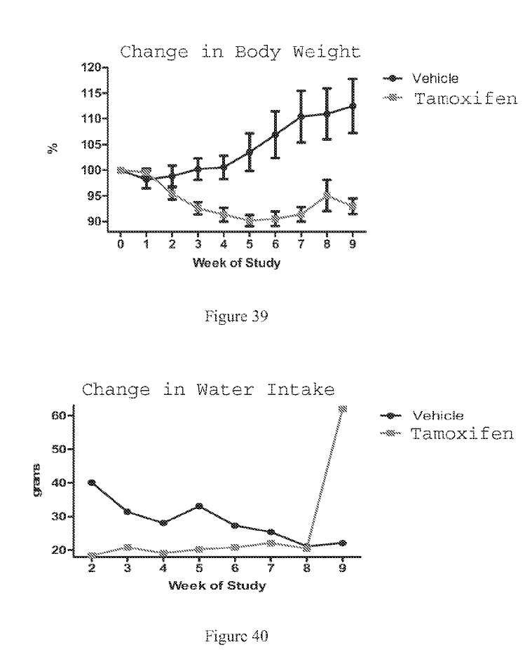

The change in body weight, water intake, food intake, fat, and lean mass for mice treated with tamoxifen are shown in FIGS. 39-43, respectively.

In this phase 2 study, the change in body weight for mice treated with clomiphene was also significant.

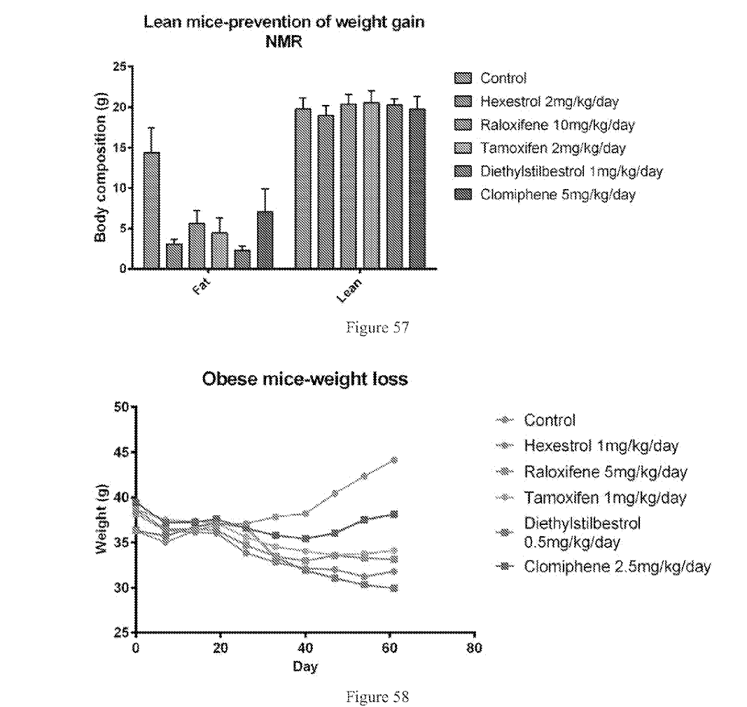

The change in body weight over time in treated and control mice is shown in FIGS. 58 and 59.

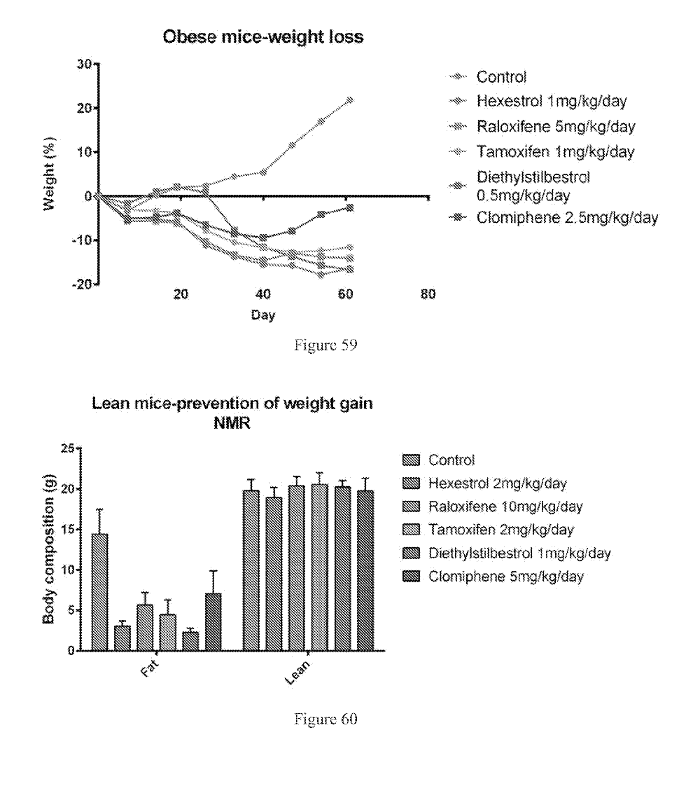

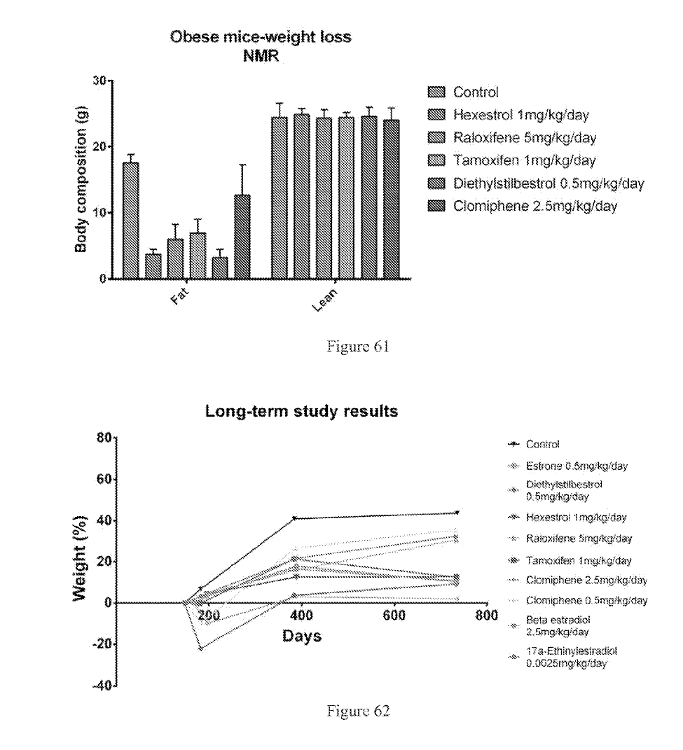

The change in food intake over time in treated and control mice is shown in FIG. 60. Food intake did not significantly change in any of the treatment groups in comparison to control mice.

FIG. 61 is a bar graph showing the change in fat and lean mass in treated and control mice after the 8-week study as measured by a NMR body composition analysis. All treated mice, except those treated with clomiphene, exhibited a statistically significant reduction in fat tissue mass in comparison to control mice. The clomiphene treated mice also exhibited a reduction in fat tissue mass. Lean tissue mass remained unchanged from control mice in all treated groups.

In this phase 2 study, the bone mineral density was significantly increased for the mice treated with these five drugs (diethylstilbestrol, hexestrol, raloxifene, tamoxifen, and clomiphene).

### Example 2

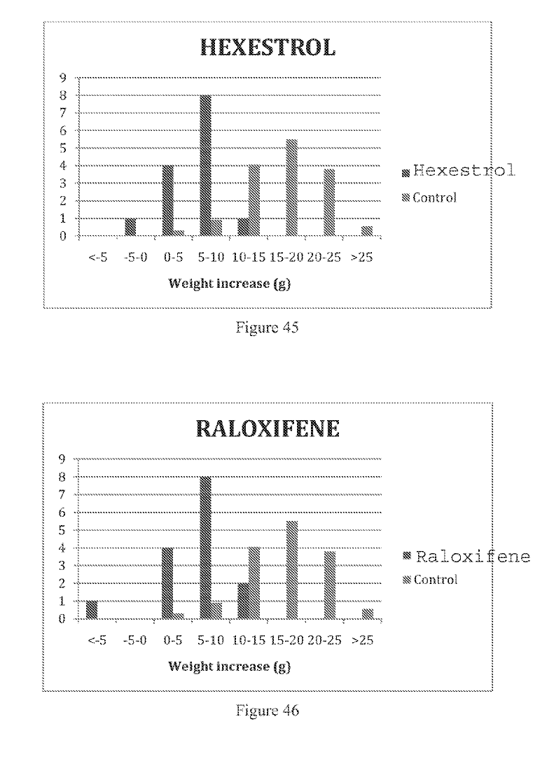

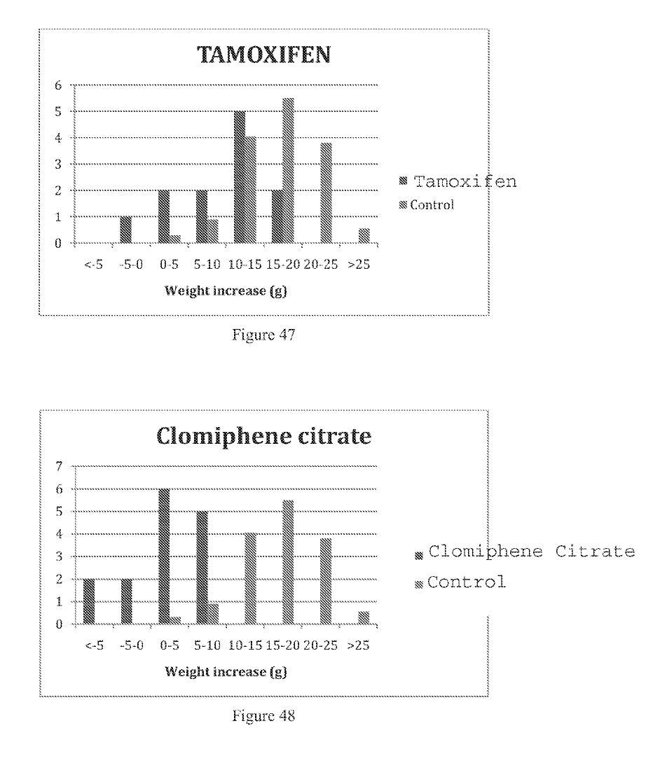

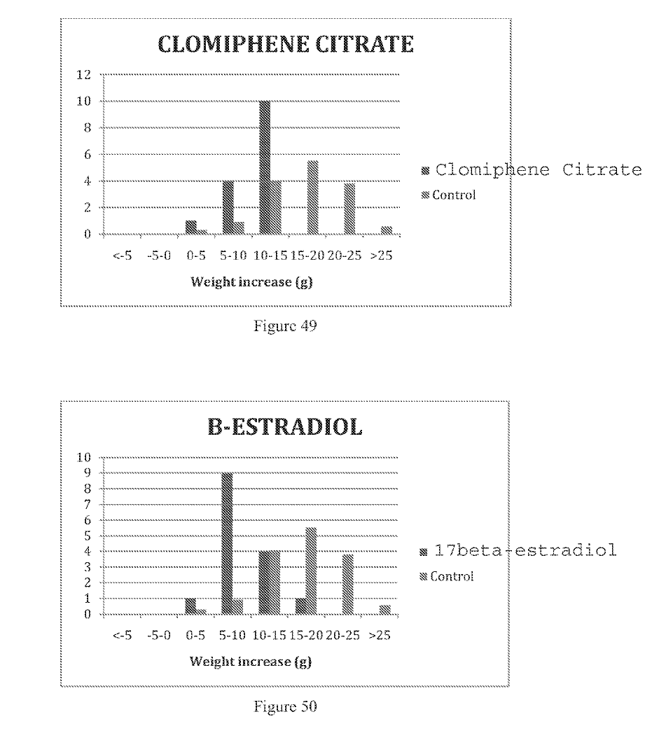

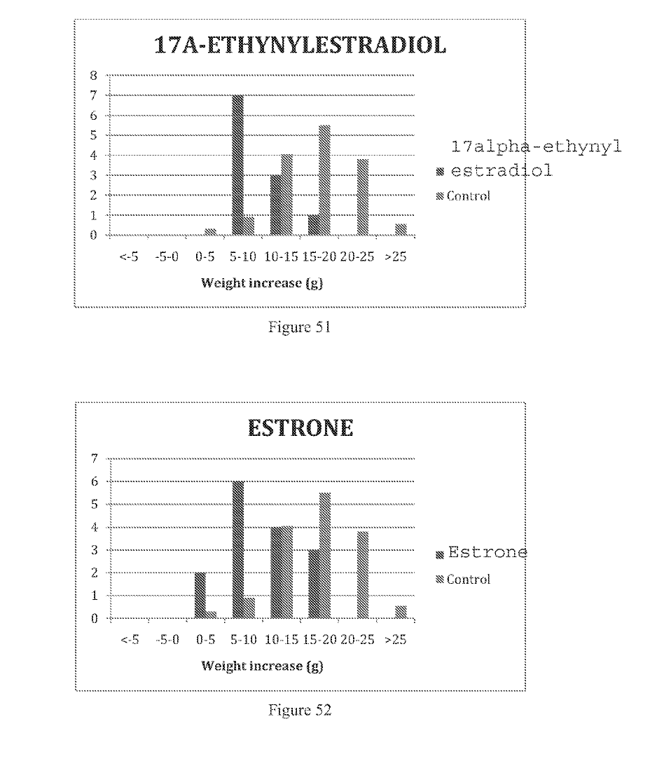

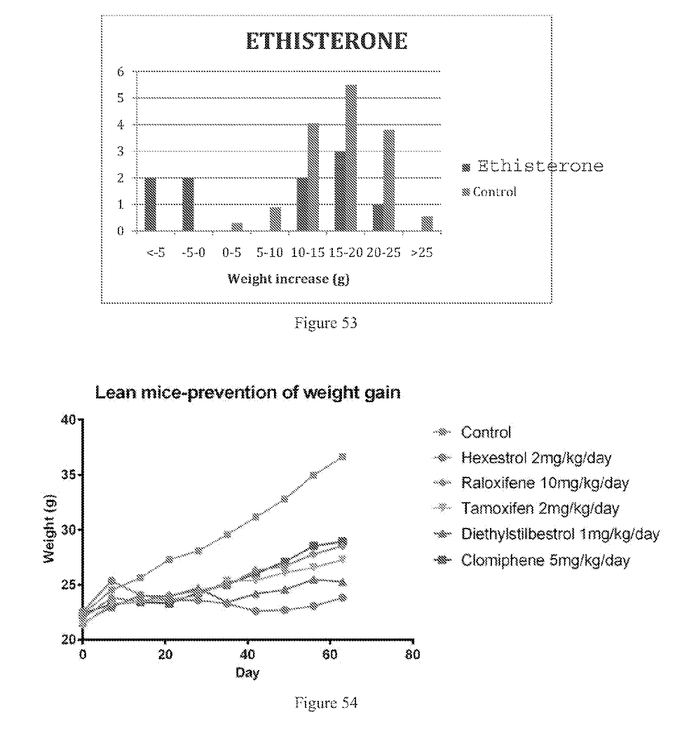

B6C3F1/J female mice (initially ˜5 months old) fed a normal diet were administered diethylstilbestrol, raloxifene, tamoxifen, clomiphene, or hexestrol daily for 1 year. The mice were grouped into cohorts of 15 mice per compound and housed at a density of 5 mice per cage. Compound treatments were provided via drinking water starting at 5 months of age and weight measurements were taken at 5 months, 6 months, 1 year, and 2 years of age. The concentration of each drug in the water is provided below. 335 mice were used as controls. The results for each drug after 1 year are shown in the figure indicated in the parenthetical to the right of the drug name.
Diethylstilbestrol: 1 mg/500 mL (0.5 mg/kg/day) (FIG. 44)
Hexestrol: 2 mg/500 mL (1 mg/kg/day) (FIG. 45)
Raloxifene: 10 mg/500 mL (5 mg/kg/day) (FIG. 46)
Tamoxifen: 2 mg/500 mL (1 mg/kg/day) (FIG. 47)
Clomiphene: 5 mg/500 mL (2.5 mg/kg/day) (FIG. 48)
Clomiphene: 1 mg/500 mL (0.5 mg/kg/day) (FIG. 49)
17β-estradiol: 2.5 mg/kg/day (FIG. 50)
17α-ethynyl estradiol: 0.025 mg/kg/day (FIG. 51)
Estrone: 0.5 mg/kg/day (FIG. 52)
Ethisterone (FIG. 53)

The results for estrone, diethylstilbestrol, hexestrol, raloxifene, tamoxifen, clomiphene, 17β-estradiol, and 17α-ethynyl estradiol are provided in FIG. 62.

The female mice treated with these drugs gained significantly less weight than the control mice (which were not administered any drug).

### Example 3

An 8 week long weight loss experiment was performed on obese 18 week old male C57BL6/J mice having been on a high fat diet for approximately 12 weeks. The estrogen modulating compound treatment groups (hexestrol, clomiphene, raloxifene, and tamoxifen) were either provided in drinking water or feed. Obese mice were allocated into cohorts of 6 mice per compound per treatment type and housed at a density of 1 mouse per cage. Water treatment group mice were placed on a high fat diet ad libitum with compound provided via drinking water. Each feed treatment group received a specially formulated high fat diet containing their corresponding compound ad libitum and normal drinking water. Weight measurements of each mouse were taken twice per week over 8 weeks. Food intake per mouse per day was also recorded.

At the end of 8 weeks mice underwent a body tissue composition analysis by Echo MRI (NMR), an intraperitoneal glucose tolerance test (IPGTT), and an oral glucose tolerance test (OGTT). For IPGTT, each mouse received an intraperitoneal glucose solution injection of 1 g glucose/kg body weight. Serum glucose concentration was measured pre-injection, and 30, 60, and 90 minutes post-injection. For OGTT, each mouse received an oral glucose solution dose of 1 g glucose/kg body weight. Serum glucose was measured before the oral dose, and 15, 30, 90, and 120 minutes after oral dose.

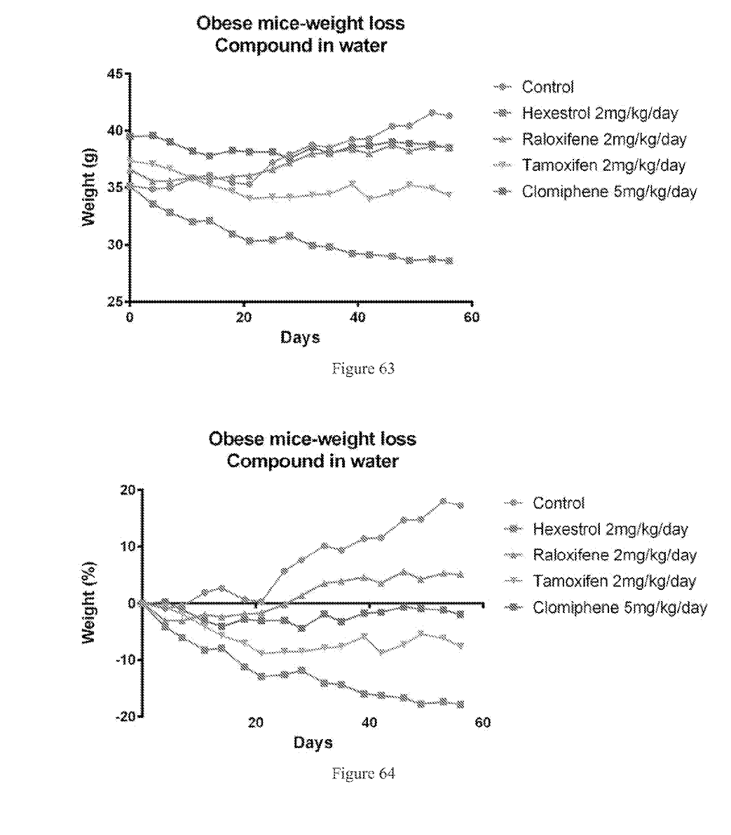

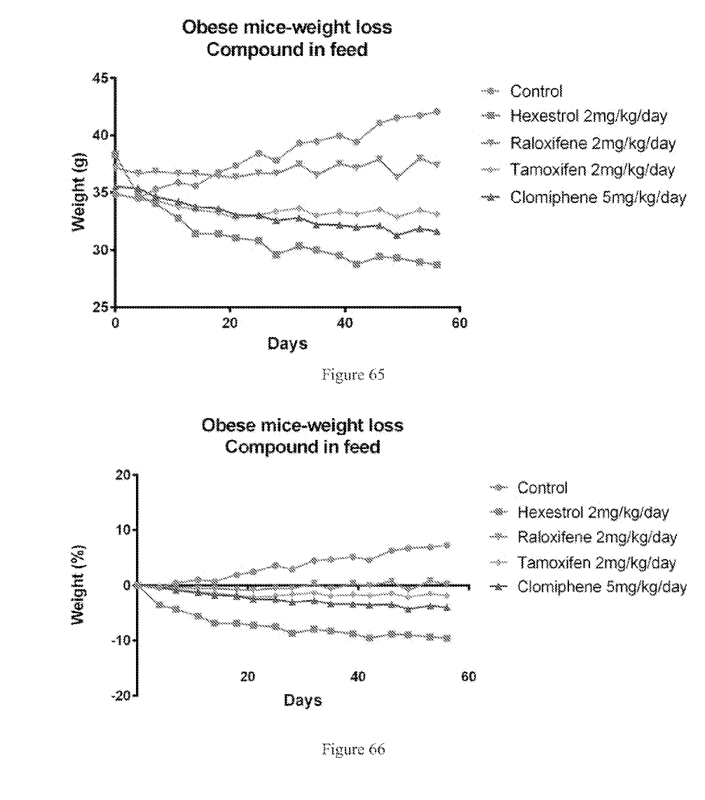

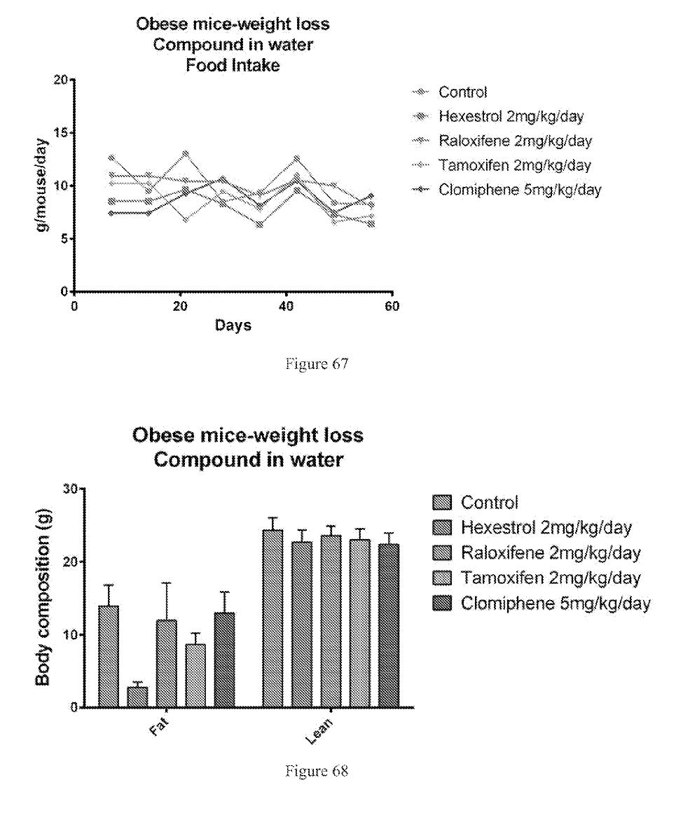

After 8 weeks of treatment, all four compounds (hexestrol, raloxifene, tamoxifen, and clomiphene) in both water and feed treatment groups resulted a significant reduction in body weight in comparison to controls, with hexestrol being the most effective with 30.8±9.7% and 31.7±4.5% body weight reduction from controls respectively. The change in body weight over time in treated and control mice is shown in FIGS. 63-66.

The change in food intake over time in treated and control mice is shown in FIG. 67. Food intake did not significantly change in any of the treatment groups in comparison to control mice.

FIGS. 68 and 69 are bar graphs showing the change in fat and lean mass in mice treated with the compound in water and feed, respectively, after the 8-week study as measured by a NMR body composition analysis. NMR body composition analysis of water treated mice indicated a significant decrease in fat tissue mass of mice treated with hexestrol and tamoxifen. Body composition analysis of feed treated mice indicated a significant decrease in fat tissue mass in hexestrol, tamoxifen, and clomiphene. In both water and feed treatment groups, hexestrol was the most effective in decreasing the amount of fat tissue. Lean tissue mass remained unchanged from controls in all groups.

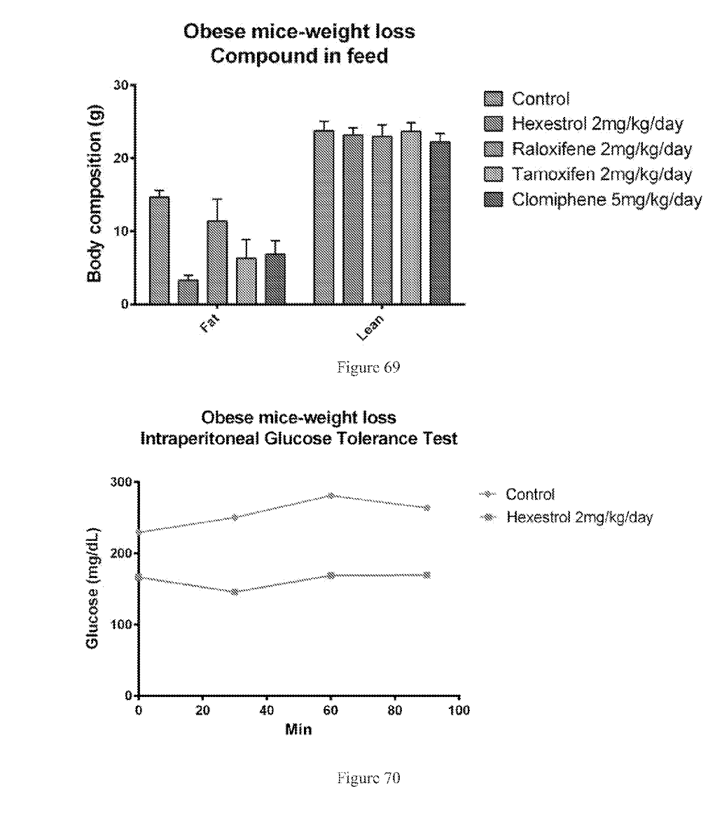

IPGTT of hexestrol water treated mice indicated that starting serum glucose concentration is significantly lower for the hexestrol group in comparison to controls. Upon the injection of the glucose solution, the hexestrol group serum glucose concentration remains static, while serum glucose of control group increases significantly by at least 18% and does not return to its starting concentration by 90 minutes post-injection. See FIG. 70.

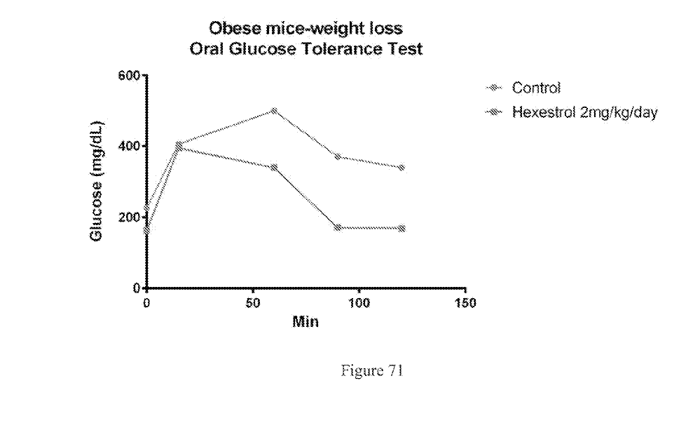

OGTT of water treated mice indicated that upon an oral dose of glucose, serum glucose concentration of hexestrol treated mice increases to a lesser amount in comparison to controls and returns back to baseline by 90 minutes post-dose, where the control group's serum glucose concentration does not return to baseline by 120 minutes post-dose. See FIG. 71.

Both glucose tolerance tests display the ability of hexestrol to improve glucose tolerance in obese mice after an 8 week treatment period in comparison to controls, which is impressive considering that attenuated glucose tolerance is an indicator of Type 2 Diabetes and Metabolic Syndrome.

### Example 4

An 8 week experiment was performed to investigate the ability of various estrogen modulating compounds to induce weight loss in obese mice. Male C57BL6/J mice were placed on a high fat diet for approximately 12 weeks prior to the study and initiated in the experiment at 18 weeks of age. Obese mice were allocated into cohorts of 6 mice per compound and housed at a density of 3 per cage. Over 8 weeks, mice were placed on a high-fat diet ad libitum and compounds were provided via drinking water. Most compounds were assigned to two treatment groups with doses of 0.5 mg/kg/day and 2.5 mg/kg/day. Hexestrol was assigned to 5 treatment groups ranging between 0.125 mg/kg/day and 2 mg/kg/day. Weight measurements of each mouse were taken once per week over 8 weeks.

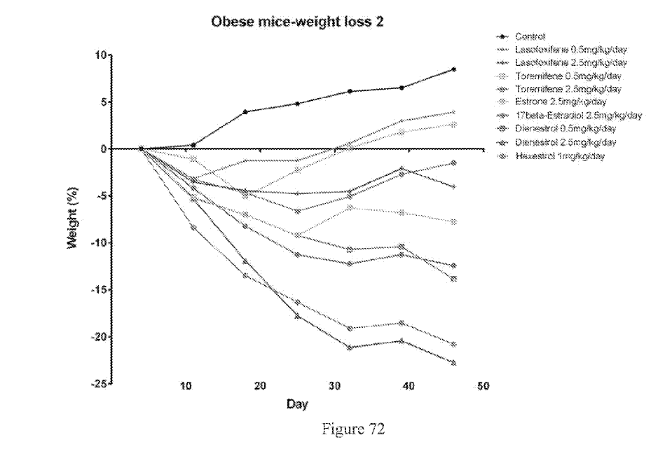

Dienestrol, hexestrol, 17β-estradiol, estrone, and toremifene all showed a significant reduction of body weight in comparison to the control group. See FIG. 72.

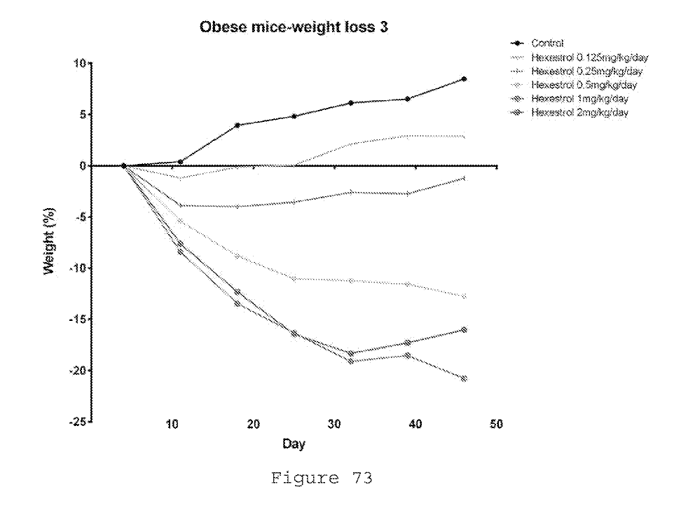

FIG. 73 shows the effects of increasing daily doses of hexestrol from 0.125 mg/kg/day to 2 mg/kg/day.

All patents, patent publications, and other references cited herein are hereby incorporated by reference in their entireties.

We claim:

1. A method of reducing body weight in a subject in need thereof comprising administering an effective amount of an estrogen receptor modulator (preferably an estrogen receptor agonist).

2. A method of reducing fat stored in adipose tissue without a substantial reduction in lean mass comprising administering an effective amount of an estrogen receptor modulator.

3. A method of treating obesity in a subject comprising administering an effective amount of an estrogen receptor modulator.

4. A method of reducing body weight in a subject in need thereof comprising administering an effective amount of (a) an estrogen receptor modulator and (b) an anti-obesity or weight loss agent.

5. A method of reducing fat stored in adipose tissue without a substantial reduction in lean mass comprising administering an effective amount of (a) an estrogen receptor modulator and (b) an anti-obesity or weight loss agent.

6. A method of treating obesity in a subject comprising administering an effective amount of (a) an estrogen receptor modulator and (b) an anti-obesity or weight loss agent.

7. A method of reducing the risk of obesity in a subject comprising administering an effective amount of an estrogen receptor modulator.

8. A method of reducing the risk of obesity or weight gain in a subject comprising administering an effective amount of (a) an estrogen receptor modulator and (b) an anti-obesity or weight loss agent.

9. The method of claim 7 or 8, wherein the subject has a propensity to become overweight or obese.

10. The method of any one of claims 7-9, wherein the subject is not overweight or obese.

11. A method of reducing the risk of a metabolic syndrome in a subject comprising administering an effective amount of an estrogen receptor modulator.

12. A method of reducing the risk of a metabolic syndrome in a subject comprising administering an effective amount of (a) an estrogen receptor modulator and (b) an anti-obesity or weight loss agent.

13. The method of claim 11 or 12, wherein the metabolic syndrome is type II diabetes.

14. A method of treating a metabolic syndrome in a subject comprising administering an effective amount of an estrogen receptor modulator.

15. A method of treating a metabolic syndrome in a subject comprising administering an effective amount of (a) an estrogen receptor modulator and (b) an anti-obesity or weight loss agent.

16. The method of claim 14 or 15, wherein the metabolic syndrome is type II diabetes.

17. A method of treating hyperglycemia in a subject in need thereof comprising administering to the subject a therapeutically effective amount of an estrogen receptor modulator.

18. A method of treating impaired glucose tolerance in need thereof comprising administering to the subject a therapeutically effective amount of an estrogen receptor modulator.

19. The method of any of the preceding claims, wherein the estrogen receptor modulator is a selective estrogen receptor modulator.

20. The method of any of the preceding claims, wherein the estrogen receptor modulator is an estrogen receptor α modulator.

21. The method of any of the preceding claims, wherein the estrogen receptor modulator is an organic compound, or a pharmaceutically acceptable salt thereof, which either possesses, or can be metabolized into a compound containing, at least one phenolic moiety, and the organic compound or its active metabolite has sufficient lipophilicity to achieve binding to an estrogen receptor.

22. The method of any of the preceding claims, wherein (a) the estrogen receptor modulator is an organic compound, or a pharmaceutically acceptable salt thereof, which either possesses, or can be metabolized into a compound containing, two phenolic moieties, (b) the phenolic moieties are separated by a moiety sufficient to achieve binding to an estrogen receptor, and (c) the organic compound or its active metabolite has sufficient lipophilicity to achieve binding to the estrogen receptor.

23. The method of claim 21 or 22, wherein the organic compound comprises one or more moieties which contain a phenolic moiety, or can be metabolized into a phenolic moiety, selected from steroidals, diphenylethylenes, triphenylethylenes, diphenylethanes, triphenylethanes, benzothoiophenes, benzopyrans, flavones, isoflavones, di-phenyl pyrazoles, tri-phenyl pyrazoles, coumestins, benzothiaphenes, benzofurans, dibenzazulenes, benzazulenes, benzopyrroles, and tetrahydronaphthylenes.

24. The method of any of the preceding claims, wherein the estrogen receptor modulator is raloxifene, hexestrol, diethylstilbestrol, tamoxifen, clomiphene, femarelle, ormeloxifene, toremifene, lasofoxifene, 17β-estradiol, 17α-ethynyl estradiol, estrone, ethisterone, dienestrol, an analog thereof, a pharmaceutically acceptable salt of any of the foregoing, or any combination of any of the foregoing.

25. The method of any of the preceding claims, wherein the estrogen receptor modulator is raloxifene or a pharmaceutically acceptable salt thereof.

26. The method of any of claims 1-24, wherein the estrogen receptor modulator is hexestrol or a pharmaceutically acceptable salt thereof.

27. The method of any of claims 1-24, wherein the estrogen receptor modulator is diethylstilbestrol or a pharmaceutically acceptable salt thereof.

28. The method of any of claims 1-24, wherein the estrogen receptor modulator is clomiphene or a pharmaceutically acceptable salt thereof.

29. The method of any of claims 1-24, wherein the estrogen receptor modulator is dienestrol or a pharmaceutically acceptable salt thereof.

30. The method of any of claims 1-24, wherein the estrogen receptor modulator is toremifene or a pharmaceutically acceptable salt thereof.

31. The method of any of claims 1-24, wherein the estrogen receptor modulator is lasofoxifene or a pharmaceutically acceptable salt thereof.

32. The method of any of the preceding claims, wherein the estrogen receptor modulator is administered intravenously.

33. The method of any of claims 1-31, wherein the estrogen receptor modulator is administered orally.

34. The method of any of claims 1-31, wherein the estrogen receptor modulator is administered transdermally.

35. The method of any of the preceding claims, wherein the subject is a human.

36. The method of any of the preceding claims, wherein the subject is a postmenopausal woman.

37. The method of any of the preceding claims, wherein the human is less than 65 years old.

38. The method of claim 37, wherein the human is less than 50 years old.

39. The method of claim 38, wherein the human is less than 40 years old.

40. The method of claim 38, wherein the human is from about 20 to about 40 years old.

41. The method of claim 38, wherein the human is a teenager.

42. The method of claim 38, wherein the human is an adolescent.

43. The method of any of the preceding claims, wherein the subject is a male.

44. The method of any of the preceding claims, wherein the subject is a pre-menopausal female.

45. The method of any of the preceding claims, wherein the subject does not suffer from osteoporosis.

46. The method of any of the preceding claims, wherein the subject does not suffer from cancer.

47. The method of any of the preceding claims, wherein the subject is overweight.

48. The method of any of claims 1-46, wherein the subject is obese.

49. The method of any of the preceding claims, wherein the subject has a propensity to become overweight or obese.

50. The method of any of the preceding claims, wherein the subject is being administered a drug associated with weight gain.

51. The method of any of the preceding claims, wherein the lean mass of the subject is not substantially reduced by the treatment.

52. The method of any of the preceding claims, wherein the food consumption of the subject is not reduced during the treatment.

53. The method of any one of claims 4-6, 8-10, 12, 13, and 15-52, wherein the anti-obesity or weight loss agent is selected from PYY (peptide YY), cholecytokinin (CCK), oxyntomodulin, ghrelin antagonist, NPY antagonist, cannabinoid CB1 receptor antagonist, a lipase inhibitor, a monoamine reuptake inhibitor, an anticonvulsant, a glucose sensitizer, a incretin mimetic, an amylin analog, a GLP-1 analog, a Y receptor peptide, a 5-HT2C serotonin receptor agonist, an opioid receptor antagonist, an appetite suppressant, an anorectic, a hormone, or a pharmaceutically acceptable salt thereof.

54. The method of any one of claims 4-6, 8-10, 12, 13, and 15-52, wherein the anti-obesity or weight loss agent is selected from rimonabant, surinabant, orlistat, cetilistat, sibutramine, bupropion, citalopram, escitalopram, fluoxetine, paroxetine, sertraline, duloxetine, milnacipran, mirtazapine, venlafaxine, des venlafaxine, tesofensine, topiramate, zonisamide, metformin, exenatide, pramlintide, liraglutide, obinepitide, lorcaserin, naltrexone, phentermine, phendimetrazine, insulin, leptin, and pharmaceutically acceptable salts thereof.

55. The method of any one of claims 4-6, 8-10, 12, 13, and 15-52, wherein the anti-obesity or weight loss agent is selected from phentermine, diethylpropion, phendimetrazine, orlistat, bupropion, topiramate, zonisamide, metformin, and pharmaceutically acceptable salts thereof.

56. The method of claim 55, wherein the anti-obesity or weight loss agent is orlistat.
